SEQUENCE LISTING (52 sequences)

SEQ ID NO: 1 - protein, 126 aa, synthetic construct
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSS

SEQ ID NO: 2 - protein, 122 aa, synthetic construct
EVQLVESGGGLVQPGGSLRLSCAASGFTFRSFAMSWVRQAPGKGLELVSDLRTSGGSTYYADSVKGRLTISRDNSKNTLYLQMNSLRAEDTAVYYCAKSHYSTSWFGGFDYWGQGTLVTVSS

SEQ ID NO: 3 - protein, 121 aa, synthetic construct
QVQLQESGPGLVKPSETLSLTCTVSGGSISSYYWSWIRQPPGKGLELIGYIYYSGSTNYNPSLKSRVTISVDTSKNHFSLKLSSVTAADTAVYYCARSQYTSSWYGSFDIWGQGTMVTVSS

SEQ ID NO: 4 - protein, 122 aa, synthetic construct
QVQLVQSGAEVKKPGASVKVSCKASGYTFNSYGISWVRQAPGQGLEWMGWISSHNGNSHYVQKFQGRVSMTTDTSTSTAYMELRSLRSDDTAVYYCARHSYTTSWYGGFDYWGQGTLVTVSS

SEQ ID NO: 5 - protein, 122 aa, synthetic construct
EVQLVESGGGLVQPGGSLRLSCAASGFTFSSYALTWVRQAPGKGLEWVSFISGSGGRPFYADSVKGRFTISRDNSKNMLYLQMNSLRAEDTAIYYCAKSLYTTSWYGGFDSWGQGTLVTVSS

SEQ ID NO: 6 - protein, 122 aa, synthetic construct
EVQLVESGGGLVQPGGSLRLSCAASGFTFSNYAMTWVRQAPGKGLEWVSTISGSGDNTYYADSVQGRFTISRGHSKNTLYLQMNSLRAEDTAVYYCAKPTYSRSWYGAFDFWGQGTMVTVSS

SEQ ID NO: 7 - protein, 122 aa, synthetic construct
EVQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSS

SEQ ID NO: 8 - protein, 116 aa, synthetic construct
EVQLLESGGGLVQPGGSLRLSCAASGFTFSDYYMNWVRQAPGKGLEWVSSISRYSSYIYYADSVKGRFTISRDNSKNTLYLQMNSLRAEDTAVYYCARDIGGMDVWGQGTLVTVSS

SEQ ID NO: 9 - protein, 119 aa, synthetic construct
EVQLLESGGGLVQPGGSLRLSCAASGFTFSNYYMHWVRQAPGKGLEWVSSISARSRYHYYADSVKGRFTISRDNSKNTLYLQMNSLRAEDTAVYYCARLATRHNAFDIWGQGTLVTVSS

SEQ ID NO: 10 - protein, 119 aa, synthetic construct
EVQLLESGGGLVQPGGSLRLSCAASGFTFSNYYMHWVRQAPGKGLEWVSSISARSSYIYYADSVKGRFTISRDNSKNTLYLQMNSLRAEDTAVYYCARLATRNNAFDIWGQGTLVTVSS

SEQ ID NO: 11 - protein, 119 aa, synthetic construct
EVQLLESGGGLVQPGGSLRLSCAASGFTFSRYYMHWVRQAPGKGLEWVSSISAQSSHIYYADSVEGRFTISRDNSKNTLYLQMNSLRAEDTAVYYCARLATRQNAFDIWGQGTLVTVSS

SEQ ID NO: 12 - protein, 119 aa, synthetic construct
EVQLLESGGGLVQPGGSLRLSCAASGFTFSNYYMHWVRQAPGKGLEWVSSISARSSYLYYADSVKGRFTISRDNSKNTLYLQMNSLRAEDTAVYYCARLATRHVAFDIWGQGTLVTVSS

SEQ ID NO: 13 - protein, 139 aa, synthetic construct
MRAWIFFLLCLAGRALAQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLFWGQGTLVTVSS

SEQ ID NO: 14 - protein, 123 aa, synthetic construct
EVQLVETGGGLIQPGGSLRLSCAASGFTFSSYAMSWVRQAPGKGLEWVSAISGSGGSTYYADSVKGRFTISRDNSKNTLYLQMNSLRAEDTAVYYCARTLHGIRAAYDAFIIWGQGTLVTVSS

SEQ ID NO: 15 - protein, 123 aa, synthetic construct
EVQLVETGGGLIQPGGSLRLSCAASGFTFSFYAMSWVRQAPGKGLEWVSAISGSGGSTYYADSVKGRFTISRDNSKNTLYLQMNSLRAEDTAVYYCARTLHGIRAAYDAFIIWGQGTLVTVSS

SEQ ID NO: 16 - protein, 123 aa, synthetic construct
EVQLVETGGGLIQPGGSLRLSCAASGFTFSFYAMSWVRQAPGKGLEWVSAISGSGGSTYYADSVKGRFTISRDNSKNTLYLQMNSLRAEDTAVYYCARTIHGIRAAYDAFIIWGQGTLVTVSS

SEQ ID NO: 17 - protein, 121 aa, synthetic construct
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGHYYYTSYSLGYWGQGTLVTVSS

SEQ ID NO: 18 - protein, 119 aa, synthetic construct
EVQLVESGGGLVQPGGSLRLSCAASGFTFSSFSMSWVRQAPGKGLEWVATISGGKTFTDYVDSVKGRFTISRDDSKNTLYLQMNSLRAEDTAVYYCTRANYGNWFFEVWGQGTLVTVSS

SEQ ID NO: 19 - protein, 106 aa, synthetic construct
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVL

SEQ ID NO: 20 - protein, 107 aa, synthetic construct
DIQMTQSPSSVSASVGDRVTITCRASQGFSSWLAWYQQKPGKAPKLLIYAASSLQSGVPSRFSGSGSGTDFTLTITNLQPEDFATYYCQQANSFPLTFGGGTKVEIK

SEQ ID NO: 21 - protein, 107 aa, synthetic construct
DIQMTQSPSSVSASVGDRVTITCRASQGISTWLAWFQQKPGKAPKLLIYAASTLQGGVPSRFSGSGSGPEFTLTISSLQPEDFATYYCQQANSFPWTFGQGTKVEIK

SEQ ID NO: 22 - protein, 107 aa, synthetic construct
DIQMTQSPSSVSASVGDRVTITCRASQGFSSWLAWYQQKPGKAPQLLIYAASSLQSGVPSRFSGSGSGSDFTLTISSLQPEDFATYYCQQANSFPLTFGGGTKVEIK

SEQ ID NO: 23 - protein, 107 aa, synthetic construct
DIQMTQSPSSVSASVGDRVTITCRASQGVVSWLAWYQQKPGKAPKLLIYAASSLQSGVPSRFSGSGSGTDFTLTISSLQPEDFATYYCQQSNSFPFTLGPGTKVDIK

SEQ ID NO: 24 - protein, 107 aa, synthetic construct
DIQMTQSPSSVSASVGDRVTITCRASQGISSWLAWYQQKPGKAPQLLIYAASRLQSGVPSRFWGSGSGTDFTLTISSLQPEDFATYYCQQANNFPFTFGPGTKVDIK

SEQ ID NO: 25 - protein, 107 aa, synthetic construct
DIQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIK

SEQ ID NO: 26 - protein, 109 aa, synthetic construct
QSVLTQPPSASGTPGQRVTISCTGSSSNIGAVYDVHWYQQLPGTAPKLLIYRNNQRPSGVPDRFSGSKSGTSASLAISGLRSEDEADYYCQTYDSSRWVFGGGTKLTVL

SEQ ID NO: 27 - protein, 110 aa, synthetic construct
QSVLTQPPSASGTPGQRVTISCSGSSSNIGNNAVSWYQQLPGTAPKLLIYASNMRVIGVPDRFSGSKSGTSASLAISGLRSEDEADYYCGAWDDSQKALVFGGGTKLTVL

SEQ ID NO: 28 - protein, 110 aa, synthetic construct
QSVLTQPPSASGTPGQRVTISCSGSSSNIGRNAVNWYQQLPGTAPKLLIYASNMRVSGVPDRFSGSKSGTSASLAISGLRSEDEADYYCWAWDDSQKVGVFGGGTKLTVL

SEQ ID NO: 29 - protein, 110 aa, synthetic construct
QSVLTQPPSASGTPGQRVTISCSGSSSNIGRNAVNWYQQLPGTAPKLLIYASNMRRSGVPDRFSGSKSGTSASLAISGLRSEDEADYYCSAWDDSQKVVVFGGGTKLTVL

SEQ ID NO: 30 - protein, 110 aa, synthetic construct
QSVLTQPPSASGTPGQRVTISCSGSSSNIGNNAVNWYQQLPGTAPKLLIYASNMRRPGVPDRFSGSKSGTSASLAISGLRSEDEADYYCEAWDDSQKAVVFGGGTKLTVL

SEQ ID NO: 31 - protein, 125 aa, synthetic construct
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIKR

SEQ ID NO: 32 - protein, 108 aa, synthetic construct
EIVLTQSPGTLSLSPGERATLSCRASQSVGINLSWYQQKPGQAPRLLIYGASHRATGIPDRFSGSGSGTDFTLTISRLEPEDFAVYYCHQYSQSPPFTFGGGTKVEIK

SEQ ID NO: 33 - protein, 108 aa, synthetic construct
EIVLTQSPGTLSLSPGERATLSCRASQSVGINLSWYQQKPGQAPRLLIYGASHRLTGIPDRFSGSGSGTDFTLTISRLEPEDFAVYYCHQYSQPPPFTFGGGTKVEIK

SEQ ID NO: 34 - protein, 108 aa, synthetic construct; identical to SEQ ID NO: 33

SEQ ID NO: 35 - protein, 106 aa, synthetic construct
DIQMTQSPSSLSASVGDRVTITCKASQNINKHLDWYQQKPGKAPKLLIYFTNNLQTGVPSRFSGSGSGTDFTLTISSLQPEDFATYYCFQYNQGWTFGGGTKVEIK

SEQ ID NO: 36 - protein, 111 aa, synthetic construct
EIVLTQSPATLSLSPGERATLSCRASESVAKYGLSLLNWFQQKPGQPPRLLIFAASNRGSGIPARFSGSGSGTDFTLTISSLEPEDFAVYYCQQSKEVPFTFGQGTKVEIK

SEQ ID NO: 37 - protein, 5 aa, synthetic construct
SYAMS

SEQ ID NO: 38 - protein, 17 aa, synthetic construct
GISAIDQSTYYADSVKG

SEQ ID NO: 39 - protein, 17 aa, synthetic construct
QKFMQLWGGGLRYPFGY

SEQ ID NO: 40 - protein, 11 aa, synthetic construct
SGEGMGDKYAA

SEQ ID NO: 41 - protein, 7 aa, synthetic construct
RDTKRPS

SEQ ID NO: 42 - protein, 9 aa, synthetic construct
GVIQDNTGV

SEQ ID NO: 43 - protein, 119 aa, synthetic construct
QVQLQQSGPELVKPGASVKTSCKASGYSFTSYYIHWVKQRPGQGLEWIGWIYPGSGNTKYNEKFKGKATLTADTSSSTAFMQLSSLTSEDSAVYYCASGFHYYGRMDYWGQGTTLTVSS

SEQ ID NO: 44 - protein, 107 aa, synthetic construct
DIQMTQSPSSLSASLGERVSLTCRASQEISGYLSWLQQKPDGTIKRLIYSTSTLDSGVPKSFSGSRSGSDYSLTISSLESEDFADYYCLQYASSPWTFGGGTKLEIK

SEQ ID NO: 45 - protein, 5 aa, synthetic construct
SYYIH

SEQ ID NO: 46 - protein, 16 aa, synthetic construct
WIYPGSGNTKYNEKFK

SEQ ID NO: 47 - protein, 10 aa, synthetic construct
GFHYYGRMDY

SEQ ID NO: 48 - protein, 11 aa, synthetic construct
RASQEISGYLS

SEQ ID NO: 49 - protein, 7 aa, synthetic construct
STSTLDS

SEQ ID NO: 50 - protein, 9 aa, synthetic construct
LQYASSPWT

SEQ ID NO: 51 - protein, 456 aa, synthetic construct
  source 1..456: note = Tozo Heavy chain
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 212 aa, synthetic construct
  source 1..212: note = Tozo Light chain
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2026/0056210 A1
(43) Pub. Date: Feb. 26, 2026

(54) METHOD OF SELECTING PATIENTS FOR TREATMENT WITH AN IL-33 AXIS ANTAGONIST

(71) Applicant: MEDIMMUNE LIMITED, Cambridge (GB)

(72) Inventors: Ian Christopher SCOTT, Cambridge (GB); Rachel Patricia MOATE, Cambridge (GB)

(21) Appl. No.: 19/104,840

(22) PCT Filed: Aug. 18, 2023
(86) PCT No.: PCT/EP2023/072807

§ 371 Date: Feb. 19, 2025

Related U.S. Application Data
(60) Provisional application No. 63/371,936, filed on Aug. 19, 2022.

(51) Int. Cl. G01N 33/68; C07K 16/24

(52) U.S. Cl. CPC G01N 33/6869; C07K 16/244; C07K 2317/21; C07K 2317/76; G01N 2333/54; G01N 2800/125; G01N 2800/52

(57) ABSTRACT

The present disclosure relates to a method of treating a subject suffering from respiratory distress or preventing respiratory distress in a subject at risk thereof with an IL-33 axis antagonist, to a method of determining whether such a subject will respond to treatment with an IL-33 axis antagonist, and to a method of selecting a subject for treatment with an IL-33 axis antagonist, by determining whether the level of IL-33/sST2 in a sample obtained from the subject is greater than or equal to a given reference level. Uses corresponding to said methods are also provided.

### CROSS-REFERENCE TO RELATED APPLICATIONS

This application claims benefit under 35 U.S.C. § 119(e) of U.S. Provisional Patent Application No. 63/371,936, filed Aug. 19, 2022, which is incorporated by reference herein in its entirety for all purposes.

### REFERENCE TO SEQUENCE LISTING SUBMITTED ELECTRONICALLY

This application incorporates by reference a Sequence Listing submitted with this application in computer readable form (CRF) as a text file entitled “IL33-601-WO-PCT Sequence Listing” created on Aug. 17, 2023 and having a size of 51,179 bytes.

### FIELD

The present disclosure relates to a method of treating a subject suffering from respiratory distress or preventing respiratory distress in a subject at risk thereof with an IL-33 axis antagonist, to a method of determining whether such a subject will respond to treatment with an IL-33 axis antagonist, and to a method of selecting a subject for treatment with an IL-33 axis antagonist, by determining whether the level of IL-33/sST2 in a sample obtained from the subject is greater than or equal to a given reference level. Uses corresponding to said methods are also provided.

### BACKGROUND

Interleukin-33 (IL-33), also known as IL-1F11, is a member of the IL-1 family of cytokines. IL-33 is a 270 amino acid protein consisting of two domains: a homeodomain and a cytokine (IL-1 like) domain. The homeodomain contains a nuclear localisation signal (NLS). IL-33 is known to exist in different forms; a reduced form (redIL-33) and an oxidised form (oxIL-33). Previous studies have shown that the reduced form is rapidly oxidised under physiological conditions to form at least one disulphide bond in the oxidised form, and that the two forms likely have different biochemical properties and biological functions.

It was previously discovered that the reduced form of IL-33 binds to ST2, and is in fact the only known ligand of the ST2 receptor expressed by immune cells including Th2 cells and mast cells. Reduced IL-33 stimulates target cells by binding ST2 and subsequently activating NFkB and MAP kinase pathways leading to production of cytokines and chemokines such as IL-4, IL-5 and IL-13 for promoting inflammation. Soluble ST2 (sST2) is thought to be a decoy receptor that prevents IL-33 signalling by binding to reduced IL-33 and forming IL-33/sST2 complexes.

More recently, it was found that the oxidised form of IL-33 also has biological activities. It was discovered that oxidised IL-33 does not bind ST2, but instead binds to the receptor for advanced glycation end products (RAGE) and signals through this alternative pathway to regulate airway epithelial remodelling.

Accordingly, there has been significant interest in IL-33 as a therapeutic target, predominantly due to the ability of what is now known about the reduced form to stimulate ST2 and result in potent inflammatory effects, but also now due to the ability of the oxidised form to stimulate RAGE-EGFR signalling that is implicated in many wide ranging diseases.

IL-33 antagonists have been developed as therapeutics to target and bind to various IL-33 forms existing within the IL-33 axis. Several antibody therapeutics are now known to target and inhibit reduced IL-33. Such antibodies include tozorakimab (binding molecule 33_640087-7B as described in WO2016/156440), etokimab (as described in WO2015/106080), itepekimab and torudokimab.

Whilst some of these antibody therapies have shown good results in the treatment of subjects suffering from IL-33 mediated diseases such as inflammatory or respiratory diseases, not all subjects react to the treatment in the same way, for some patients the treatment with such therapies is more beneficial than others.

Tracking and monitoring how subjects respond to such therapies which target the IL-33 axis has been difficult as there has historically been no reliably sensitive IL-33 assay which can distinguish between the different forms of IL-33. An assay has now been developed which can detect such forms of IL-33, and can inform users about the biological pathways that may be inhibited or activated in a given biological sample, and about the relationship to the status of patients. The highly sensitive assay can detect not only one, but each of the different forms of IL-33; reduced IL33, oxidised IL33, and also bound IL33/sST2 in a biological sample.

Using this assay, the inventors have further discovered for the first time that patients suffering from respiratory distress can be stratified into those that are more likely to experience a beneficial therapeutic effect by treatment with such IL-33 axis antagonist therapies, and those that are less likely to experience a therapeutic effect. The inventors have surprisingly discovered that the levels of the IL-33/sST2 complex in a biological sample is indicative of the therapeutic effect of an IL-33 axis antagonist therapy. The inventors believe that measuring IL-33/sST2 in a biological sample can inform medical treatment choices and determine the likelihood of response to a IL-33 axis antagonist therapy such as those mentioned above.

Accordingly, one or more aspects of the present disclosure and further features thereof are defined hereinbelow.

### SUMMARY OF THE DISCLOSURE

According to a first aspect of the present disclosure there is provided a method of treating a subject suffering from respiratory distress or preventing respiratory distress in a subject at risk thereof, comprising administering to the subject an effective amount of an IL-33 axis antagonist, wherein the level of IL-33/sST2 in a sample obtained from the subject is greater than or equal to a reference level of about 25 pg/ml.

According to an alternative first aspect of the present disclosure there is provided an IL-33 axis antagonist for use in the treatment of a subject suffering from respiratory distress, or for use in preventing respiratory distress in a subject at risk thereof, wherein the level of IL-33/sST2 in a sample obtained from the subject is greater than or equal to a reference level of about 25 pg/ml. In one instance, the subject has a level of IL-33/sST2 greater than or equal to the reference level of about 25 pg/ml. In one instance, the subject has a baseline level of IL-33/sST2 greater than or equal to the reference level of about 25 pg/ml. In one instance, the subject has a baseline serum level of IL-33/sST2 greater than or equal to the reference level of about 25 pg/ml.

According to a second aspect of the present disclosure, there is provided a method of treating a subject suffering from respiratory distress, or preventing respiratory distress in a subject at risk thereof, wherein the method comprises: measuring the level of IL-33/sST2 in a sample obtained from the subject; and administering to the subject an effective amount of an IL-33 axis antagonist if the level of IL-33/sST2 in the sample is greater than or equal to a reference level of about 25 pg/ml.

According to an alternative second aspect of the present disclosure, there is provided an IL-33 axis antagonist for use in a method of treating a subject suffering from respiratory distress, or for use in preventing respiratory distress in a subject at risk thereof, wherein the method comprises: measuring the level of IL-33/sST2 in a sample obtained from the subject; and administering to the subject an effective amount of an IL-33 axis antagonist if the level of IL-33/sST2 in the sample is greater than or equal to a reference level of about 25 pg/ml.

According to a third aspect of the present disclosure there is provided a method of preventing acute respiratory failure in a subject at risk thereof, comprising administering to the subject an effective amount of an IL-33 axis antagonist, wherein the level of IL-33/sST2 in a sample obtained from the subject is greater than or equal to a reference level of about 25 pg/ml.

According to an alternative third aspect of the present invention, there is provided an IL-33 axis antagonist for use in preventing acute respiratory failure in a subject at risk thereof, wherein the level of IL-33/sST2 in a sample obtained from the subject is greater than or equal to a reference level of about 25 pg/ml.

In one instance the methods of the third aspect may comprise measuring the level of IL-33/sST2 in a sample obtained from the subject; and administering to the subject an effective amount of an IL-33 axis antagonist if the level of IL-33/sST2 in the sample is greater than or equal to a reference level of about 25 pg/ml.

In one instance the subject at risk thereof in the methods of the third aspect is a subject suffering from respiratory distress.

According to a fourth aspect of the present disclosure, there is provided a method of reducing the likelihood of death and/or acute respiratory failure in a subject suffering from respiratory distress or at risk of suffering from respiratory distress, comprising administering to the subject an effective amount of an IL-33 axis antagonist, wherein the level of IL-33/sST2 in a sample obtained from the subject is greater than or equal to a reference level of about 25 pg/ml.

According to an alternative fourth aspect of the present invention, there is provided an IL-33 axis antagonist for use in reducing the likelihood of death and/or acute respiratory failure in a subject suffering from respiratory distress or at risk of suffering from respiratory distress, wherein the level of IL-33/sST2 in a sample obtained from the subject is greater than or equal to a reference level of about 25 pg/ml.

In one instance the methods of the fourth aspect may comprise measuring the level of IL-33/sST2 in a sample obtained from the subject; and administering to the subject an effective amount of an IL-33 axis antagonist if the level of IL-33/sST2 in the sample is greater than or equal to a reference level of about 25 pg/ml.

In one instance the methods of the fourth aspect comprise significantly reducing the likelihood of death and/or respiratory failure, suitably significantly reducing the likelihood of acute respiratory failure. Suitably reducing the likelihood of death and/or respiratory failure in comparison to a subject who has not received an effective amount of an IL-33 axis antagonist.

In one instance of the methods of the fourth aspect, reducing the likelihood may comprise reducing the rate of death and/or respiratory failure in subjects suffering from respiratory distress or at risk of suffering from respiratory distress.

In one instance the methods of the fourth aspect may comprise treating a subject suffering from respiratory distress by reducing the likelihood of death and/or acute respiratory failure.

In one instance, the methods may comprise methods of selectively treating a subject suffering from respiratory distress or selectively preventing respiratory distress in a subject. Suitably wherein the methods may comprise a step of selectively administering an effective amount of an IL-33 axis antagonist if the level of IL-33/sST2 in a sample obtained from the subject is greater than or equal to a reference level of about 25 pg/ml.

According to a fifth aspect of the present disclosure there is provided a method of selecting a subject suffering from respiratory distress, or at risk of suffering from respiratory distress, for treatment with an IL-33 axis antagonist comprising: measuring the level of IL-33/sST2 in a sample obtained from the subject; and selecting the subject for said treatment if the level of IL-33/sST2 in the sample is greater than or equal to a reference level of about 25 pg/ml.

In one instance, the method may comprise selecting a subject suffering from respiratory distress, or at risk of suffering from respiratory distress, who would respond to, or benefit from, treatment with an IL-33 axis antagonist.

In another instance, the method may comprise identifying a subject suffering from respiratory distress, or at risk of suffering from respiratory distress, for treatment with an IL-33 axis antagonist. In another instance, the method may comprise identifying a subject suffering from respiratory distress, or at risk of suffering from respiratory distress, who would respond to, or benefit from, treatment with an IL-33 axis antagonist.

In one instance, the method further comprises a step of administering to the subject an effective amount of an IL-33 axis antagonist.

In an alternative fifth aspect of the present disclosure, there is provided a method of selecting a subject for treatment with an IL-33 axis antagonist comprising: measuring the level of IL-33/sST2 in a sample obtained from the subject; and selecting the subject for said treatment if the level of IL-33/sST2 in the sample is greater than or equal to a reference level of about 25 pg/ml.

In one instance, the method may comprise selecting a subject who would respond to, or benefit from, treatment with an IL-33 axis antagonist.

In another instance, the method may comprise identifying a subject for treatment with an IL-33 axis antagonist. In another instance, the method may comprise identifying a subject who would respond to, or benefit from, treatment with an IL-33 axis antagonist.

In one instance, the subject is suffering from a disease, disorder, condition or infection. In one instance, the subject is suffering from respiratory distress, or at risk of suffering from respiratory distress, suitably which may be caused by a disease, disorder, condition or infection.

In one instance, the method further comprises a step of administering to the subject an effective amount of an IL-33 axis antagonist.

According to a sixth aspect of the present disclosure there is provided a method of determining whether a subject suffering from respiratory distress, or at risk of suffering from respiratory distress, is likely to respond to treatment with an IL-33 axis antagonist comprising: measuring the level of IL-33/sST2 in a sample obtained from the subject; and determining that the subject is likely to respond to said treatment if the measured level of IL-33/sST2 in the sample is greater than or equal to the reference level.

In one instance, the method may comprise determining whether a subject suffering from respiratory distress, or at risk of suffering from respiratory distress, will respond to treatment with an IL-33 axis antagonist. In one instance, the method may comprise determining whether a subject suffering from respiratory distress, or at risk of suffering from respiratory distress, is likely to benefit from treatment with an IL-33 axis antagonist. In one instance, the method may comprise determining whether a subject suffering from respiratory distress, or at risk of suffering from respiratory distress, will benefit from treatment with an IL-33 axis antagonist.

In one instance, the method further comprises a step of administering to the subject an effective amount of an IL-33 axis antagonist.

In an alternative sixth aspect of the present disclosure, there is provided a method of determining whether a subject is likely to respond to treatment with an IL-33 axis antagonist comprising: measuring the level of IL-33/sST2 in a sample obtained from the subject; and determining that the subject is likely to respond to said treatment if the measured level of IL-33/sST2 in the sample is greater than or equal to the reference level.

In one instance, the method may comprise determining whether a subject will respond to treatment with an IL-33 axis antagonist. In one instance, the method may comprise determining whether a subject is likely to benefit from treatment with an IL-33 axis antagonist.

In one instance, the method may comprise determining whether a subject will benefit from treatment with an IL-33 axis antagonist.

In one instance, the subject is suffering from a disease, disorder, condition or infection. In one instance, the subject is suffering from respiratory distress, or at risk of suffering from respiratory distress, suitably which may be caused by a disease, disorder, condition or infection.

In one instance, the method further comprises a step of administering to the subject an effective amount of an IL-33 axis antagonist.

In one instance, the methods of the sixth aspects may comprise determining or predicting the likelihood that a subject suffering from respiratory distress, or at risk of suffering from respiratory distress, will respond to, or benefit from, treatment with an IL-33 axis antagonist.

Suitably wherein the methods comprise determining or predicting that the subject has an increased likelihood of responding to, or benefiting from, said treatment if the measured level of IL-33/sST2 in the sample is greater than or equal to the reference level. Suitably an increased likelihood relative to a reference subject as defined herein.

In one instance of any of the methods, the IL-33 axis antagonist comprises a heavy chain variable region having VHCDRs 1-3 of SEQ ID NO: 37, SEQ ID NO: 38 and SEQ ID NO: 39, respectively, and a light chain variable region having VLCDRs 1-3 of SEQ ID NO: 40, SEQ ID NO: 41 and SEQ ID NO: 42, respectively. In one instance of any of the methods, the IL-33 axis antagonist is tozorakimab.

In one instance of any of the methods, the subject is suffering from acute respiratory failure, or is at risk of acute respiratory failure.

In one instance any of the methods may comprise a step of comparing the measured level of IL-33/sST2 in the sample with the reference level of about 25 pg/ml.

In one instance, the measuring step of any of the methods may comprise assaying a sample obtained from the subject, suitably to measure the level of IL-33/sST2. In one instance, any of the methods may comprise the steps of (a) contacting the sample with one or more binding molecules capable of binding to IL-33/sST2 under conditions sufficient to form complexes; and (b) detecting the level of IL-33/sST2 complexes in the sample.

For the avoidance of doubt, each aspect or instance of a “method” of treatment includes compositions for use in said method of treatment and the use of said composition in the manufacture of a medicament for use in said methods of treatment.

### Definitions

‘IL-33’ protein as employed herein refers to interleukin 33, in particular a mammalian interleukin 33 protein, for example human protein deposited with UniProt number 095760. However, given the present inventors findings, it clear that this entity is not a single species but instead exists as reduced and oxidized forms. The terms “IL-33” and “IL-33 polypeptide” are used interchangeably. In certain instances, IL-33 is full length. In another instance, IL-33 is mature, truncated IL-33 (amino acids 112-270). Recent studies suggest full length IL-33 is active (Cayrol and Girard, Proc Natl Acad Sci USA 106(22): 9021-6 (2009); Hayakawa et al., Biochem Biophys Res Commun. 387(1):218-22 (2009); Talabot-Ayer et al, J Biol Chem. 284(29): 19420-6 (2009)). However, N-terminally processed or truncated IL-33 including but not limited to aa 72-270, 79-270, 95-270, 99-270, 107-270, 109-270, 111-270, 112-270 may have enhanced activity (Lefrancais 2012, 2014). In another instance, IL-33 may include a full length IL-33, a fragment thereof, or an IL-33 mutant or variant polypeptide, wherein the fragment of IL-33 or IL-33 variant polypeptide retains some or all functional properties of active IL-33.

‘Oxidized IL-33’ or ‘oxIL-33’ is a protein visible as a distinct band, for example by western blot analysis under non-reducing conditions, in particular with a mass 4 Da less than the corresponding reduced from. In particular, it refers to a protein with one or two disulphide bonds between the cysteines independently selected from cysteines 208, 227, 232 and 259.

In one instance, oxidized IL-33 shows no binding to ST2. ‘Reduced IL-33’ or ‘redIL-33’ as employed herein refers to the form of the IL-33 that binds to ST2 and triggers ST2 mediated signalling. In particular cysteines 208, 227, 232 and 259 of the reduced form are not disulfide bonded.

‘IL-33/sST2’ as employed herein refers to the form of reduced IL-33 that is bound to soluble ST2 receptors. ‘IL-33/sST2’ as used herein may equally be referred to as an ‘IL-33/sST2 complex’.

‘Antigenically distinct forms of IL-33’ as employed herein refers to any form of IL-33 which can act as an antigen and be discriminated by an antibody or binding fragment thereof, typically in the context of the present disclosure this means oxidised IL-33, reduced IL-33 and reduced IL-33/sST2 complexes.

It is to be noted that the term “a” or “an” entity refers to one or more of that entity; for example, “an anti-IL-33 antibody” is understood to represent one or more anti-IL-33 antibodies. As such, the terms “a” (or “an”), “one or more,” and “at least one” can be used interchangeably herein.

As used herein, the terms “treat” or “treatment” refer to both therapeutic treatment and prophylactic or preventative measures, i.e. prevention, wherein the object is to prevent or slow down (lessen) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The term “prevention” is used herein to mean prevent, delay or treat, or all, as appropriate, development or continuance or aggravation of a condition or disorder.

By “subject” or “individual” or “animal” or “patient” or “mammal,” is meant any subject, particularly a mammalian subject, for whom diagnosis, prognosis, or therapy is desired, except where the subject is defined as a ‘healthy subject’. Mammalian subjects include humans; domestic animals; farm animals; such as dogs, cats, guinea pigs, rabbits, rats, mice, horses, cattle, cows, and so on.

The term “assaying” is used to refer to the act of identifying, screening, probing, testing measuring or determining, which act may be performed by any conventional means. The term “detecting” (and the like) means the act of extracting particular information from a given source, which may be direct or indirect. In some instances of the methods disclosed herein, the presence of IL-33/sST2 (e.g., level of, etc.) is detected in a biological sample. The terms “assaying” and “determining” contemplate a transformation of matter, e.g., a transformation of a biological sample, e.g., a serum sample, from one state to another by means of subjecting that sample to physical testing.

The term “obtaining” means to procure, e.g., to acquire possession of in any way, e.g., by physical intervention (e.g., biopsy) or non-physical intervention (e.g., transmittal of information via a server), etc.

The term “pharmaceutical composition” is defined herein to refer to a mixture or solution containing at least one therapeutic agent to be administered to a subject, e.g., a mammal or human, in order to prevent or treat a particular disease or condition.

The term “pharmaceutically acceptable” is defined herein to refer to those compounds, materials, compositions and/or dosage forms, which are, within the scope of sound medical judgment, suitable for contact with the tissues a subject, e.g., a mammal or human, without excessive toxicity, irritation allergic response and other problem complications commensurate with a reasonable benefit/risk ratio.

As used herein, “selecting” or “identifying” in reference to a subject is used to mean that a particular subject is specifically chosen from a larger group of subjects on the basis of (due to) the particular subject having a predetermined criteria, e.g., equal to or greater than a reference level of IL-33/sST2. Similarly, “selectively treating” refers to providing treatment to a subject, where that subject is specifically chosen from a larger group of subjects on the basis of the particular subject having a predetermined criteria, e.g., equal to or greater than a reference level of IL-33/sST2. Similarly, “selectively administering” refers to administering a drug to a subject that is specifically chosen from a larger group of subjects on the basis of (due to) the particular subject having a predetermined criteria, e.g., a particular biological marker such as equal to or greater than a reference level of IL-33/sST2. By selecting, identifying, selectively treating and selectively administering, it is meant that a subject is delivered a personalised therapy based on the patient's particular biology, rather than being delivered a standard treatment regimen based solely on the subject having or being at risk of a particular disease or condition. Selecting, in reference to a method of treatment as used herein, does not refer to fortuitous treatment of a subject that may happen to have equal to or greater than the reference level of IL-33/sST2, but rather refers to the deliberate choice to administer an IL-33 axis antagonist to a subject based on the subject having equal to or greater than the reference level of IL-33/sST2. Thus, selective treatment differs from standard treatment, which delivers a particular drug to all subjects, regardless of this biomarker.

As used herein, “predicting” or “determining” indicates that the methods described herein provide information to enable a health care provider to ascertain the likelihood that an individual having equal to or greater than a reference level of IL-33/sST2 will respond to or will respond more favourably to treatment with an IL-33 axis antagonist. It does not refer to the ability to predict response with 100% accuracy. Instead, the skilled artisan will understand that it refers to an increased probability.

As used herein, “likelihood” and “likely” is a measurement of how probable an event is to occur. It may be used interchangeably with “probability”. Likelihood refers to a probability that is more than speculation, but less than certainty. Thus, an event is likely if a reasonable person using common sense, training or experience concludes that, given the circumstances, an event is probable. The phrase “increased likelihood” refers to an increase in the probability that an event will occur. For example, some methods herein allow prediction of whether a subject will display an increased likelihood of responding to treatment with an IL-33 axis antagonist in comparison to a reference subject who does not have equal to or greater than a certain reference level of IL-33/sST2.

Further features of the above defined aspects are described hereinbelow in headed sections. Each section is combinable with any of the above mentioned aspects in any compatible combination.

### DETAILED DESCRIPTION

### Subject

The methods of the disclosure relate to treating a subject, determining if a subject will benefit from treatment, and selecting a treatment for a subject. The methods each involve using a sample obtained from the subject, suitably within which the level of IL-33/sST2 is measured.

Suitably the subject may be a human. The subject may be undergoing medical care, or may be requesting medical care. Suitably the subject is male or female. Suitably the subject is an adult or a child.

Suitably the subject is suffering from respiratory distress, or is believed to be suffering from respiratory distress. Suitably the subject may have a condition, disease, disorder or infection, or may be believed to have a condition, disease, disorder or infection. Suitably the condition, disease, disorder or infection causes respiratory distress, as explained further hereinbelow.

Suitably the subject may have one or more symptoms consistent with respiratory distress such as a higher than normal breathing rate, low blood oxygen concentration, laboured breathing, shortness of breath, low blood pressure, higher than normal heart rate, chest pain, skin colour changes, sweating, wheezing, confusion, tiredness and the like.

Suitably therefore, the subject may be a patient.

Alternatively, the subject may be one believed to be at risk of respiratory distress, suitably at risk of developing such condition, disease, disorder or infection which causes respiratory distress. For example, the subject who is at risk may already have a condition, disease, disorder or infection which causes respiratory distress, or may have been in contact with an individual suffering from such a condition, disease, disorder or infection which causes respiratory distress, may suffer from a related condition, disease, disorder or infection which causes respiratory distress, or may satisfy risk factors associated with such a condition, disease, disorder or infection like smoking, old age, allergy etc. Further definitions of a subject who is at risk of respiratory distress are provided hereinbelow.

### Respiratory Distress

The methods of the disclosure relate to subjects suffering from respiratory distress, or subjects at risk of respiratory distress.

Suitably a subject suffering from respiratory distress may be defined as a subject who is unable to ventilate adequately to provide sufficient oxygen to the blood and systemic organs.

Suitably the subject suffering from respiratory distress has one or more of the following symptoms: a higher than normal breathing rate, low blood oxygen concentration, laboured breathing, shortness of breath, low blood pressure, higher than normal heart rate, chest pain, skin colour changes, sweating, wheezing, confusion, and tiredness. Suitably respiratory distress may be defined as a subject who has at least one of the following symptoms: a higher than normal breathing rate, low blood oxygen concentration, laboured breathing, shortness of breath, and may additionally comprise one or more of the following symptoms: low blood pressure, higher than normal heart rate, chest pain, skin colour changes, sweating, wheezing, confusion, and tiredness.

Suitably a subject who is at risk of respiratory distress is likely to develop one or more of the following symptoms: a higher than normal breathing rate, low blood oxygen concentration, laboured breathing, shortness of breath, low blood pressure, higher than normal heart rate, chest pain, skin colour changes, sweating, wheezing, confusion, and tiredness. Suitably a subject who is at risk of respiratory distress may have few such symptoms, or no such symptoms, but may be at risk of developing further symptoms. Suitably a subject who is at risk of respiratory distress may have a disease, disorder, condition or infection as identified elsewhere herein which is associated with respiratory distress, or which is likely to lead to respiratory distress.

Suitably a subject suffering from respiratory distress may be a subject that requires oxygen or a subject that requires ventilation. In one instance, the subject suffering from respiratory distress requires supplemental oxygen. In one instance, the subject requiring supplemental oxygen may have oxygen saturation (SpO₂) of ≤90%. In one instance the subject requiring supplemental oxygen may have SpO₂ of ≤92% and viral lower respiratory tract infection (or disease—“vLRTD”). In some instances, the vRLTD is determined from the presence of radiographic infiltrates by chest X-ray/CT scan that are compatible with viral lung infection. In another instance, the subject requiring supplemental oxygen may have SpO₂ of ≤92% and is using accessory muscles of respiration or has a respiration rate of >22/minute.

In one instance the subject suffering from respiratory distress is hospitalized. Suitably before the methods of the invention are carried out. Suitably the subject may be suffering from respiratory failure, or may be at risk of respiratory failure, suitably acute respiratory failure (ARF) or chronic respiratory failure (CRS). Suitably the respiratory failure may be type 1 (Hypoxemic), Type 2 (Hypercapnic), Type 3 (Peri-operative), or Type 4 (shock induced).

In one instance, the subject is suffering from acute respiratory failure (ARF), or is at risk of acute respiratory failure (ARF). In one instance, the subject is suffering from hypoxemic (Type 1) acute respiratory failure, or is at risk of hypoxemic (Type 1) acute respiratory failure. In one instance, the subject is suffering from hypercapnic (Type 2) acute respiratory failure, or is at risk of hypercapnic (Type 2) acute respiratory failure.

Suitably a subject who is at risk of respiratory failure, acute or otherwise, may be a subject having any one or more of the above symptoms of respiratory distress, such as a higher than normal breathing rate, low blood oxygen concentration, laboured breathing, shortness of breath, low blood pressure, higher than normal heart rate, chest pain, skin colour changes, sweating, wheezing, confusion, and tiredness.

Suitably a subject who is at risk of respiratory failure, acute or otherwise, is a subject suffering from respiratory distress. Suitably respiratory distress, may typically lead to respiratory failure if left untreated. In one instance therefore, the subject may be suffering from respiratory distress and at risk of developing respiratory failure, suitably acute respiratory failure.

Suitably respiratory distress, such as acute respiratory failure (ARF), may be caused by a condition, disease, disorder or infection. Suitably the condition, disease, disorder or infection is a respiratory disease. Suitably the respiratory disease is a disease which affects the trachea, bronchi, bronchioles, alveolar ducts and/or alveoli.

Suitably respiratory distress, such as acute respiratory failure (ARF), may be caused by any of the following conditions, diseases, or disorders: acute respiratory distress syndrome (ARDS), chronic obstructive pulmonary disease (COPD), asthma, bronchitis, bronchiectasis, emphysema, heart failure, myocardial ischemia, mitral stenosis, pulmonary oedema, pulmonary embolism, thromboembolism, cystic fibrosis, amylotophic lateral sclerosis, muscular dystrophy, Guillain-Barre syndrome, myasthenia gravis, poliomyelitis, polymyositis, botulism, hypokalemia, hypophosphatemia, myxedema, hypothyroidism, sepsis, stroke, acute pancreatitis, transfusion, reperfusion, drug or alcohol overdose, trauma to the chest, viral or bacterial infection, inhalation injury (from inhaling smoke, fumes, or chemicals), aspiration, near drowning. Suitably therefore the subject may have any of these conditions, diseases, or disorders. Suitably the subject many have any of these conditions, diseases, or disorders and is at risk of respiratory distress, such as acute respiratory failure (ARF).

In one instance the subject has asthma. In one instance the subject has COPD.

Suitably respiratory distress, such as acute respiratory failure (ARF), is caused by a bacterial or viral infection, suitably a bacterial or viral respiratory infection, suitably a bacterial or viral lower respiratory tract infection. Suitably therefore the subject may have a bacterial or viral infection, suitably a bacterial or viral lower respiratory tract infection. Suitably the subject may have a bacterial or viral infection, suitably a bacterial or viral lower respiratory tract infection, and is at risk of respiratory distress, such as acute respiratory failure (ARF).

In one instance, the subject suffering from respiratory distress has, or is suspected of having, a viral lower respiratory tract infection or disease.

Suitable bacterial or viral respiratory infections may be selected from: tonsillitis, scarlet fever, pharyngitis, laryngitis, diphtheria, angina, Lemmiere syndrome, tularemia, the plague, enterocolitis, common cold, influenza, mononucleosis, HIV infection, pneumonia, suitably viral pneumonia, bronchitis, psittacosis, SARS, MERS, and COVID-19.

Suitably such infections may be caused by the following bacteria or viruses: Streptococcus sp., Arcanobacterium haemolyticum, Neisseria gonorrhoeae, Corynebacterium diphtheriae, Fusobacterium necrophorum, Francisella tulareniss, Yersinia pestis, Yersinia enterocolitica, Adenovirus sp., herpes simplex virus (HSV), HIV, Coxsackievirus sp., Coronavirus sp., Rhinovirus sp., Influenza A or B viruses, Parainfluenza viruses, Bocaparvovirus sp., Metapneumovirus sp., Respiratory syncytial virus (RSV), Epstein Barr virus, Cytomegalovirus sp., Mycoplasma pneumoniae, Chlamydophla pneumoniae, and Chhlmaydophla psittaci.

In one instance, respiratory distress, such as acute respiratory failure (ARF), is caused by a Coronavirus infection. Suitably therefore the subject may have a coronavirus infection. Suitably the subject may have a coronavirus infection and is at risk of respiratory distress, such as acute respiratory failure (ARF). Suitably the Coronavirus infection may be an alphacoronavirus infection or a betacoronavirus infection. Suitably the alphacoronavirus infection may be an infection of the following viruses: Alphacoronavirus 1 (TGEV, Feline coronavirus, Canine coronavirus), Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, and Scotophilus bat coronavirus 512. Suitably the betacoronavirus infection may be an infection of any of the following viruses: Betacoronavirus 1 (Bovine Coronavirus, Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-CoV, SARS-CoV-2), and Tylonycteris bat coronavirus HKU4.

In one instance, the Coronavirus infection is a SARS infection, suitably a SARS-CoV-2 infection. In one instance, respiratory distress, such as acute respiratory failure (ARF), is caused by COVID-19 derived from a SARS-CoV-2 infection. In one instance, the subject is suffering from respiratory distress or acute respiratory failure caused by COVID-19, or a SARS-CoV-2 infection. In one instance, the subject is suffering from COVID-19 and is at risk of respiratory distress, or at risk of acute respiratory failure.

In one instance, the subject is suffering from or has COVID-19, or a SARS-CoV-2 infection. In one instance, the subject is at risk of COVID-19 or at risk of having a SARS-CoV-2 infection.

In one instance, the respiratory distress, such as acute respiratory failure (ARF), is caused by an Influenza A or B virus, a Parainfluenza virus, a Coronavirus or Respiratory syncytial virus infection. In one instance, the respiratory distress, such as acute respiratory failure (ARF), is caused by viral pneumonia derived from infection with any of an Influenza A or B virus, a Parainfluenza virus, a Coronavirus or Respiratory syncytial virus. Suitably therefore, the subject may have an Influenza A or B virus, a Parainfluenza virus, a Coronavirus or Respiratory syncytial virus infection and is at risk of respiratory distress, such as acute respiratory failure (ARF). Suitably therefore, the subject may have an Influenza A or B virus, a Parainfluenza virus, a Coronavirus or Respiratory syncytial virus infection and is at risk of viral pneumonia.

In one instance, the subject is suffering from respiratory distress or acute respiratory failure caused by pneumonia, suitably viral pneumonia. In one instance, the subject is suffering from or has pneumonia, suitably viral pneumonia. In one instance, the subject at risk of pneumonia or viral pneumonia.

In one instance, the viral pneumonia is caused by COVID-19, suitably derived from infection with a Coronavirus which may be selected from any of those listed above, suitably from infection with SARS-CoV-2. In some instances, the pneumonia is caused by influenza virus A, influenza virus B, respiratory syncytial virus, human parainfluenza virus, adenovirus, metapneumovirus, SARS-COV, Middle East respiratory syndrome virus (MERS-CoV), hantavirus, herpes simplex virus, varicella-zoster virus, measles virus, rubella virus, cytomegalovirus, smallpox virus or dengue virus. In some instances, the pneumonia is caused by influenza virus A, influenza virus B, respiratory syncytial virus or human parainfluenza virus.

Suitably therefore, in some instances, the subject is suffering from, or has, both COVID-19 and viral pneumonia. In some instances, wherein the viral pneumonia is caused by COVID-19, or a SARS-CoV-2 infection. In some instances, the subject is suffering from COVID-19 and is at risk of viral pneumonia.

Suitably in any of the instances above, the subject has tested positive for SARS-CoV-2 infection. Suitably any available tests in the art may be used to determine if the subject has a SARS-CoV-2 infection, such as antigen tests, antibody tests or nucleic acid based tests.

Suitably nucleic acid based tests comprise PCR tests. Suitably any of the methods of the disclosure may further comprise a step of testing the subject for the presence of SARS-CoV-2 infection or for the presence of COVID-19.

Suitably any references to subjects having any condition, disease, disorder or infection herein may also be references to subjects who are at risk of having said condition, disease, disorder or infection. Suitably an IL-33 axis antagonist may also be used to prevent conditions, diseases, disorders or infections in subjects at risk thereof, wherein the level of IL-33/sST2 in a sample obtained from the subject is greater than or equal to a reference level of about 25 pg/ml

Suitably therefore the disclosure further provides methods of preventing said conditions, diseases, disorders or infections in subjects who are at risk thereof. Suitably said methods of preventing comprise administering to the subject an effective amount of an IL-33 axis antagonist describe elsewhere herein.

Suitably as identified above, a subject may be at risk of suffering from respiratory distress, or at risk of acute respiratory failure. Suitably therefore the disclosure provides methods of preventing respiratory distress in a subject at risk thereof, or preventing death and/or acute respiratory failure in a subject at risk thereof, wherein the level of IL-33/sST2 in a sample obtained from the subject is greater than or equal to a reference level of about 25 pg/ml.

In one instance there is provided a method of preventing acute respiratory failure in a subject at risk thereof, comprising administering to the subject an effective amount of an anti-IL-33 antibody or antigen binding fragment thereof comprising a heavy chain variable region having VHCDRs 1-3 of SEQ ID NO: 37, SEQ ID NO: 38 and SEQ ID NO: 39, respectively, and a light chain variable region having VLCDRs 1-3 of SEQ ID NO: 40, SEQ ID NO: 41 and SEQ ID NO: 42, respectively, wherein the level of IL-33/sST2 in a sample obtained from the subject is greater than or equal to a reference level of about 25 pg/ml.

In one instance there is provided a method of preventing acute respiratory failure in a subject suffering from acute respiratory distress, comprising administering to the subject an effective amount of an anti-IL-33 antibody or antigen binding fragment thereof comprising a heavy chain variable region having VHCDRs 1-3 of SEQ ID NO: 37, SEQ ID NO: 38 and SEQ ID NO: 39, respectively, and a light chain variable region having VLCDRs 1-3 of SEQ ID NO: 40, SEQ ID NO: 41 and SEQ ID NO: 42, respectively, wherein the level of IL-33/sST2 in a sample obtained from the subject is greater than or equal to a reference level of about 25 pg/ml.

In one instance there is provided a method of preventing acute respiratory failure in a subject suffering from COVID-19, pneumonia, or viral pneumonia, comprising administering to the subject an effective amount of an anti-IL-33 antibody or antigen binding fragment thereof comprising a heavy chain variable region having VHCDRs 1-3 of SEQ ID NO: 37, SEQ ID NO: 38 and SEQ ID NO: 39, respectively, and a light chain variable region having VLCDRs 1-3 of SEQ ID NO: 40, SEQ ID NO: 41 and SEQ ID NO: 42, respectively, wherein the level of IL-33/sST2 in a sample obtained from the subject is greater than or equal to a reference level of about 25 pg/ml.

Suitably the disclosure also provides methods of reducing the likelihood of death and/or acute respiratory failure in a subject at risk thereof, suitably in a subject suffering from respiratory distress. Suitably the disclosure also provides methods of reducing the rate of death and/or acute respiratory failure in subjects at risk thereof, suitably in subjects suffering from respiratory distress.

In one instance of any of the methods described herein, the subject has, or is suspected of having, a viral lower respiratory tract infection, and optionally may be hospitalized, and/or optionally may require supplemental oxygen or ventilation. In one instance of any of the methods described herein, the subject has a viral lower respiratory tract infection, is hospitalized, and requires supplemental oxygen or ventilation.

### Sample

The present disclosure relies on measuring the level of IL-33/sST2 in a sample taken from the subject. Suitably the sample may be a test sample.

Suitably the sample is a biological sample. The term “biological sample” as used herein refers to a sample from a subject, which may be used for the purpose of identification, diagnosis, prediction, or monitoring. Suitably, the sample is a body fluid sample. Alternatively, suitable samples may include tissue samples, such as biopsies.

A suitable body fluid sample may include: a blood sample (for example, a whole blood sample, a blood plasma sample, or a serum sample or a combination thereof); a mucosal lining fluid sample (for example mucosal lining fluid from an epithelium); a lavage sample (for example a lung or bronchoalveolar lavage sample); or a supernatant sample (for example from a culture of epithelium cells).

Suitably a supernatant sample may be an epithelial cell supernatant sample. Suitably from a bronchial epithelial culture. Suitably the supernatant may be from the cells or from the air-liquid interface of the culture.

Suitably the mucosal fluid lining samples or lavage samples contain biological cells, suitably these samples contain epithelium cells.

Suitably IL-33/sST2 is prevalent in serum. Therefore, in one instance, the sample is a serum sample.

Suitably the methods of the disclosure may further comprise a step of obtaining a sample. Suitably obtaining a sample from a subject. Suitably this step takes place prior to measuring the level of IL-33/ST2 in the sample. Alternatively, the sample may have previously been obtained, prior to carrying out the methods of the disclosure.

Suitably the sample is obtained and the level of IL-33/ST2 in the sample is measured prior to any treatment of the subject. Suitably therefore, it is the baseline level of IL-33/sST2 in the sample that is measured.

Suitably a blood sample, may be taken by a blood draw from the subject. Suitably a serum sample may be obtained from a blood sample by coagulating the blood and centrifuging the blood to obtain the serum supernatant.

Suitably a mucosal lining fluid sample may be taken by a nasal swab from the subject.

Suitably a lavage sample may be taken by a lung wash from the subject.

Suitably a supernatant sample may be taken by culturing cells from the subject. Suitably by culturing epithelium cells isolated from the epithelium of a subject. Suitably isolated from the respiratory epithelium of a subject.

Suitably, the sample is representative of cytokine levels in the subject in respect of whom the methods of the disclosure are being practised. Suitably, the sample is representative of interleukin levels in a subject in respect of whom the method is being practised. Suitably, the sample is representative of IL-33 levels in a subject in respect of whom the method is being practised. Suitably, the sample is representative of IL-33/sST2 levels in a subject in respect of whom the method is being practised. Suitably, the sample is representative of baseline IL-33/sST2 levels in a subject in respect of whom the method is being practised.

Samples may be processed for the enrichment of IL-33/sST2. Suitable techniques for such enrichment may be determined with reference to the nature of the sample. Generally, examples of suitable techniques (such as techniques for the isolation of biological molecules from a sample) will be well known to those skilled in the art. Suitably the sample may be filtered, purified, concentrated, or the like.

### Measuring IL-33/sST2

The methods of the disclosure may comprise step of measuring the level of IL-33/sST2 in a sample obtained from a subject, or refer to subjects having certain level of IL-33/sST2 which has been measured in a sample therefrom.

Suitably by ‘level’ it is meant any suitable quantification of the amount of IL-33/sST2 in a sample. Suitably the ‘level’ may mean the mass, volume, or concentration of IL-33/sST2 in a sample. Suitably the level means the concentration of IL-33/sST2 in a sample. Suitably the concentration may be measured in an suitable units such as: international units per millilitre (IU/ml), grams per millilitre (g/ml), % w/v, Moles (M). In one instance, the concentration is measured in grams per millilitre (g/ml), suitably in micrograms per millilitre (ug/ml), nanograms per millilitre (ng/ml), picograms per millilitre (pg/ml), femtograms per millilitre (fg/ml)). In one instance, the concentration of IL-33/sST2 is measured in picograms per millilitre (pg/ml).

Suitably, as explained hereinabove, the level of IL-33/sST2 is the baseline level of IL-33/sST2. Suitably the baseline serum level of IL-33/sST2. In one instance, the subject has a level of IL-33/sST2 greater than or equal to the reference level of about 25 pg/ml. In one instance, the subject has a baseline level of IL-33/sST2 greater than or equal to the reference level of about 25 pg/ml. In one instance, the subject has a baseline serum level of IL-33/sST2 greater than or equal to the reference level of about 25 pg/ml.

Suitably the level of IL-33/sST2 is measured in a sample obtained from the subject of interest. Suitably any method of measuring IL-33/sST2 in a sample may be used. Suitably any method which is capable of determining the level of IL-33/sST2 in a sample.

Examples of suitable samples are described elsewhere herein. It will be appreciated that the nature of a sample may then determine the nature of assay techniques that may be used in practicing a method of the disclosure.

IL-33/sST2 is formed from two proteins bound together in a complex. Suitably therefore the assay technique is a protein assay technique. Suitably therefore the methods of the disclosure may comprise a step of measuring IL-33/sST2 in a sample obtained from the subject using an assay, suitably assaying a sample obtained from the subject to determine the level of IL-33/sST2 in the sample.

Protein levels can be determined by various assay techniques such as S-plex, ELISA, radioimmunoassay, immunoprecipitation, Western blot and mass spectrometry. Such assay techniques will be known to the person skilled in the art.

Suitably the level of IL-33/sST2 in a sample obtained from a subject is determined by an immunoassay. Suitably therefore the methods of the disclosure may comprise a step of measuring the level of IL-33/sST2 in a sample obtained from the subject using an immunoassay, suitably performing an immunoassay on a sample obtained from the subject to determine the level of IL-33/sST2 in the sample.

Immunoassays typically require capture reagents, such as antibodies, to capture the relevant analyte, and optionally probe reagents, to detect the relevant analyte. Suitable immunoassay techniques are: ELISA (enzyme linked immunosorbent assay), S-plex, western blotting, immunocytochemistry, immunoprecipitation, affinity chromotography, Bio-Layer Interferometry, Octet, ForteBio) and biochemical assays such as Dissociation-Enhanced Lanthanide Fluorescent Immunoassays (DELFIA®, Perkin Elmer), Forster resonance energy transfer (FRET) assays (e.g. homogeneous time resolved fluorescence (HTRF®, Cis Biointernational), and radioimmuno/radioligand binding assays.

Western blot analysis generally comprises preparing protein samples, electrophoresis of the protein samples in a polyacrylamide gel (e.g., 8-20 SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding Western blot protocols see, e.g., Ausubel et al., eds, (1994) Current Protocols in Molecular Biology (John Wiley & Sons, Inc., NY) Vol. 1 at 10.8.1.

ELISAs comprise preparing antigen, coating the well of a 96-well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well.

One of skill in the art would be knowledgeable as to the parameters that can be modified to increase the signal detected as well as other variations of ELISAs known in the art. For further discussion regarding ELISAs see, e.g., Ausubel et al., eds, (1994) Current Protocols in Molecular Biology (John Wiley & Sons, Inc., NY) Vol. 1 at 13.2.1.

Suitably the level of IL-33/sST2 in a sample is determined by immunoassay. Suitably by using a modified ELISA called an S-plex assay. S-plex assays are available from Meso Scale Diagnostics LLC with suitable instructions for use.

Immunoassays will typically comprise a step of incubating a sample, in the presence of one or more binding molecules capable of specifically binding to the protein of interest, and a step of detecting the bound molecules by any of a number of techniques well known in the art. The assay described herein follows this general format.

Suitably the level of IL-33/sST2 is measured in a sample by an assay, suitably an immunoassay, which may comprise the following steps:
(a) contacting the sample with one or more binding molecules capable of binding to IL-33/sST2 under conditions sufficient to form complexes;
(b) Detecting the level of IL-33/sST2 complexes in the sample

Optionally, the method may comprise a step (c) of contacting the complexes with one or more reporter molecules capable of binding to the one or more complexes. In such an instance, the detecting step (b) comprises detecting the levels of bound reporter molecules in said sample.

Suitably therefore any of the methods of the disclosure may further comprise steps (a) and (b) above, and optional step (c).

Suitably therefore in one instance, the method of treating a subject suffering from respiratory distress, or preventing respiratory distress in a subject at risk thereof may comprise: (a) measuring the level of IL-33/sST2 in a sample obtained from the subject by (i) contacting the sample with one or more binding molecules capable of binding to IL-33/sST2 under conditions sufficient to form complexes and (ii) detecting the level of IL-33/sST2 complexes in the sample; and (b) administering to the subject an effective amount of an IL-33 axis binding antagonist if the level of IL-33/sST2 in the sample is greater than or equal to a reference level of about 25 pg/ml.

Suitably therefore in one instance, the method of selecting a subject suffering from respiratory distress, or at risk of suffering from respiratory distress, for treatment with an IL-33 axis antagonist may comprise: (a) measuring the level of IL-33/sST2 in a sample obtained from the subject by (i) contacting the sample with one or more binding molecules capable of binding to IL-33/sST2 under conditions sufficient to form complexes and (ii) detecting the level of IL-33/sST2 complexes in the sample; and (b) selecting the subject for said treatment if the level of IL-33/sST2 in the sample is greater than or equal to a reference level of about 25 pg/ml.

Suitably therefore in one instance, the method of determining whether a subject suffering from respiratory distress, or at risk of suffering from respiratory distress, is likely to respond to treatment with an IL-33 axis binding antagonist may comprise: (a) measuring the level of IL-33/sST2 in a sample obtained from the subject by (i) contacting the sample with one or more binding molecules capable of binding to IL-33/sST2 under conditions sufficient to form complexes and (ii) detecting the level of IL-33/sST2 complexes in the sample; and (b) determining that the subject is likely to respond to said treatment if the measured level of IL-33/sST2 in the sample is greater than or equal to the reference level.

Suitably any binding molecule which specifically binds to IL-33/sST2 may be used. Suitably any reporter molecule which specifically binds to IL-33/sST2-binding molecule complexes may be used.

Alternatively, the reporter molecule and binding molecule are the same entity. In such instances, the binding molecule binds directly to IL-33/sST2 and is capable of indicating as such. Suitably therefore the binding molecule may be labelled. Suitably the binding molecule may be detectably labelled.

Suitably the one or more reporter molecules, if present, are capable of indicating that they are present and/or bound. Suitably the one or more reporter molecules are labelled. Suitably the one or more reporter molecules are detectably labelled.

Suitably the binding molecules are contacted with the biological sample for a sufficient time to form complexes. Suitably the reporter molecules, if present, are contacted with the biological sample for a sufficient time to bind to the one or more complexes.

Suitably this time may be termed an incubation time. Suitably, incubation times will vary depending on the binding molecules used and optionally the reporter molecules used, as well as the biological sample.

Suitably a sufficient incubation time may be between 5 minutes and 180 minutes, suitably between 10 minutes and 150 minutes, suitably between 20 minutes and 120 minutes, suitably between 30 minutes and 100 minutes, suitably between 45 minutes and 90 minutes, suitably around 60 minutes.

Suitably the one or more binding molecules are contacted with the biological sample for around 120 minutes.

Suitably the one or more reporter molecules, if present, are contacted with the complexes/biological sample for around 120 minutes.

Suitably contacting the biological sample with the binding molecules or reporter molecules if present comprises bringing the binding molecules or reporter molecules and the biological sample together such that binding can occur.

Suitably the biological sample may be contacted with the binding molecules or reporter molecules, if present, in any suitable way using known assay techniques.

The sample can be brought in contact with the one or more binding molecules by using a solid phase support or carrier. Suitably, either the sample or binding molecules may be immobilised onto the support or carrier.

Suitably, the support may then be washed. Suitably the support is washed with a suitable buffer. Suitably the methods of the disclosure may comprise one or more steps of washing.

Suitably washing takes place after the addition of the one or more binding molecules and before addition of the one or more reporter molecules, if present. Suitably after the addition of the one or more reporter molecules, if present, and before the detecting step.

Suitably at least one washing step occurs after step (a), and suitably before step (b).

Suitably, the support may be contacted with the one or more reporter molecules after step (a). Suitably therefore, the step of contacting the complexes with one or more reporter molecules capable of binding to the one or more complexes may comprise contacting the complexes immobilised on a support with one or more reporter molecules capable of binding to the one or more complexes. Suitably the support may then be washed a second time to remove unbound reporter molecules. Suitably therefore a second washing step may occur after contacting the complexes with the one or more reporter molecules.

Optionally the binding molecules or reporter molecules, if present, may comprise a detectable label or they may be subsequently labelled. Suitable labels are discussed elsewhere herein.

The amount of bound label, which may be bound on the solid support, may then be detected by conventional means, suitably as recited in step (b).

By “solid support or carrier” it is intended to mean any support capable of binding an antigen or an antibody. Well-known supports or carriers include glass, polystyrene, polypropylene, polyethylene, dextran, nylon, nitrocellulose, amylases, natural and modified celluloses, polyacrylamides, and magnetite. The nature of the carrier can be either soluble to some extent or insoluble for the purposes of the present disclosure. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

Suitably the reporter molecules, if present, are detected by detection of a detectable label associated with each reporter molecule. Suitably the detectable label may be part of the reporter molecule or may be added separately, suitably after the reporter molecule has bound to the one or more IL-33/sST2 complexes in the sample.

Alternatively, detecting the one or more IL-33/sST2 complexes in a sample may comprise detecting the levels of the one or more bound binding molecules in said sample. In such an instance, suitably the binding molecules are directly detected by detection of a detectable label associated with each binding molecule. Suitably, in such an instance, no reporter molecules are required. Suitably the detectable label may be part of the binding molecule or may be added separately, suitably after the binding molecule has bound to the IL-33/sST2 in the sample.

Suitable detectable labels may be a radiolabel, a fluorescent label, an enzyme, a chromophore. Suitably the label may only be detectable upon stimulation. Suitable sources of stimulation will vary depending on the label used, for example in the use of an enzyme label the source of stimulation may be a substrate, in the use of a chromophore label the source of stimulation may be radiation of a particular wavelength. Such sources of stimulation may be regarded as stimulation agents.

Suitable fluorescent labels may include: rhodamine, fluorescein, Cy5, ruthenium diimine complexes, and phosphorescent porphyrin dyes.

Suitable enzyme labels may include: peroxidase, glucose oxidase, alkaline phosphatase (AP), β-galactosidase, catalase or luciferase. The substrates to be used with the specific enzymes are generally chosen for the production, upon hydrolysis by the corresponding enzyme, of a detectable colour change. For example, p-nitrophenyl phosphate is suitable for use with alkaline phosphatase conjugates; for peroxidase conjugates, 1,2-phenylenediamine or toluidine are commonly used. Suitably fluorogenic substrates may be used, which yield a fluorescent product.

Alternatively, the binding molecules or reporter molecules, if present, may not be labelled. In such an instance, the binding molecules or reporter molecules may be capable of being bound by a label, suitably a detectable label. Suitably the detectable label is capable of binding the binding molecules or reporter molecule and indicating as such. Suitably the detectable label is specific to a corresponding binding molecule or reporter molecule.

Suitably therefore the assay method may further comprise a step of adding a detectable label to the sample. Suitably, step (b) may comprise detecting the detectable label associated with the one or more binding molecules.

Alternatively, step (b) may comprise detecting the detectable label associated with the one or more reporter molecules, if present.

In one instance, the reporter molecules comprise a detectable label conjugated thereto. In such an instance, the reporter molecules are detected by detection of the conjugated detectable label.

In one instance, the one or more reporter molecules are detectably labelled. In one instance, the one or more reporter molecules comprise a detectable label conjugated thereto. In one instance, the one or more reporter molecules are sulfo-tagged.

In one instance, the binding molecules comprise a detectable label conjugated thereto. In such an instance, the binding molecules are detected by detection of the conjugated detectable label.

Suitably the assay used is highly sensitive. Suitably the assay is capable of detecting IL-33/sST2 in a sample at a concentration of microgram/ml or lower, nanogram/ml or lower, picogram/ml or lower, femtogram/ml or lower. In one instance, the assay is capable of detecting IL-33/sST2 in a sample at a concentration of picogram/ml.

Suitably the lower limit of detection is a concentration of microgram/ml or lower, nanogram/ml or lower, picogram/ml or lower, femtogram/ml or lower. Suitably the lower limit of detection is a concentration of femtogram/ml.

### Binding Molecule and Reporter Molecule

Suitably the level of IL-33/sST2 may be measured in a sample obtained from the subject by performing an assay, suitably an immunoassay comprising the steps described hereinabove.

Suitably the binding molecule and the reporter molecule for use in the assay are selected from: an antibody, an antigen-binding fragment thereof, an aptamer, at least one heavy or light chain CDR of a reference antibody molecule, and at least six CDRs from one or more reference antibody molecules.

In one instance, the binding molecules and reporter molecules are antibodies or antigen binding fragments thereof.

Suitably, the binding molecule is an anti-IL-33/sST2 antibody or binding fragment thereof. Suitably, the reporter molecules is an anti-IL-33/sST2-binding molecule complex antibody or binding fragment thereof.

“Antibody” as employed herein refers to an immunoglobulin molecule as discussed below in more detail, in particular a full-length antibody or a molecule comprising a full-length antibody, for example a DVD-Ig mole and the like.

A “binding fragment thereof” is interchangeable with “antigen binding fragment thereof” and refers to an epitope/antigen binding fragment of an antibody fragment, for example comprising a binding region, in particular comprising 6 CDRs, such as 3 CDRs in heavy variable region and 3 CDRs in light variable region.

Suitably, the antibody or binding fragment thereof is selected from: naturally-occurring, polyclonal, monoclonal, multispecific, mouse, human, humanized, primatized, or chimeric. Suitably, the antibody or binding fragment thereof may be an epitope-binding fragment, e.g., Fab′ and F(ab′)2, Fd, Fvs, single-chain Fvs (scFv), disulfide-linked Fvs (sdFv), fragments comprising either a VL or VH domain, or fragments produced by a Fab expression library. Suitably, the antibody or binding fragment thereof may be a minibody, a diabody, a triabody, a tetrabody, or a single chain antibody. Suitably, the antibody or binding fragment thereof is a monoclonal antibody. ScFv molecules are known in the art and are described, e.g., in U.S. Pat. No. 5,892,019.

Immunoglobulin or antibody molecules of the disclosure can be of any type (e.g., IgG, IgE, IgM, IgD, IgA, and IgY), class (e.g., IgGI, IgG2, IgG3, IgG4, IgAI, and IgA2, etc.), or subclass of immunoglobulin molecule.

Suitably the antibody or binding fragment thereof used as a binding molecule is a capture antibody.

Suitably the antibody or binding fragment thereof used as a reporter molecule is a probe antibody.

Suitably, the binding molecule and reporter molecule are paired, therefore each capture antibody has a corresponding probe antibody. Suitably the capture antibodies are paired with probe antibodies.

Suitable binding and reporter molecules having such capabilities, such as antibodies, are available in the art and may be ordered from reagent companies such as: R&D systems, for example.

However, suitably the binding molecules and/or reporter molecules used in the assay comprise the following sequences.

### IL33/sST2 Binding Molecule—AB1070008

Suitably the binding molecule which binds to IL-33/sST2 may comprise 3 CDRs, for example in a heavy chain variable region (VH) according to SEQ ID NO:43 and/or may comprise 3 CDRs in a light chain variable region (VL) according to SEQ ID NO:44.

Suitably the binding molecule which binds to IL-33/sST2 comprises 3 CDRs in a heavy chain variable region wherein the VH has an amino acid sequence at least 90%, for example 91, 92, 93, 94, 95, 96, 97, 98, 99 or 100% identical to a VH according to SEQ ID NO: 43, and/or comprises 3 CDRs in a light chain variable region wherein the VL has an amino acid sequence at least 90%, for example 91, 92, 93, 94, 95, 96, 97, 98, 99 or 100% identical to a VL according to SEQ ID NO: 44.

Suitably the binding molecule which binds to IL-33/sST2 may comprise a variable heavy domain (VH) and a variable light domain (VL) having VHCDRs 1-3 of: SEQ ID NO: 45, 46 and 47 wherein one or more VHCDRs have 3 or fewer single amino acid substitutions, deletions or insertions.

Suitably, 3, 2, 1, or no single amino acid substitutions, deletions or insertions.

Suitably the binding molecule which binds to IL-33/sST2 may comprise a variable heavy domain (VH) and a variable light domain (VL) having VLCDRs 1-3 of: SEQ ID NO: 48, 49 and 50 wherein one or more VLCDRs have 3 or fewer single amino acid substitutions, deletions or insertions.

Suitably, 3, 2, 1, or no single amino acid substitutions, deletions or insertions.

Suitably the binding molecule which binds to IL-33/sST2 may comprise a VH having VHCDRs 1-3 of: SEQ ID NO: 45, 46 and 47, and a VL having VLCDRs of SEQ ID NO: 48, 49 and 50.

Suitably the binding molecule which binds to IL-33/sST2 may comprise a VHCDR1 having the sequence of: SEQ ID NO:45 a VHCDR2 having the sequence of: SEQ ID NO:46 a VHCDR3 having the sequence of: SEQ ID NO:47 a VLCDR1 having the sequence of: SEQ ID NO:48 a VLCDR2 having the sequence of: SEQ ID NO:49 and a VLCDR3 having the sequence of: SEQ ID NO:50.

Suitably therefore, the binding molecule which binds to IL-33/sST2 is an antibody or binding fragment thereof comprising a VH and VL, wherein the VH has an amino acid sequence at least 90%, for example 91, 92, 93, 94, 95, 96, 97, 98, 99 or 100% identical to a VH according to SEQ ID NO: 43.

Suitably therefore, the binding molecule which binds to IL-33/sST2 is an antibody or binding fragment thereof comprising a VH and VL, wherein the VL has an amino acid sequence at least 90%, for example 91, 92, 93, 94, 95, 96, 97, 98, 99 or 100% identical to a VL according to SEQ ID NO: 44.

Suitably therefore, the binding molecule which binds to IL-33/sST2 is an antibody or binding fragment thereof comprising a VH and a VL, wherein the VH has an amino acid sequence consisting of SEQ ID NO:43, and the VL has an amino acid sequence consisting of SEQ ID NO: 44.

sST2 Reporter Molecule

Suitably the reporter molecule which binds to sST2 may be obtained from any suitable supplier. For example, suitably the reporter molecule which binds to sST2 is MAB5232 from R&D Systems. Other examples of sST2 binding molecules that may be useful in the assays disclosed herein are MAB523, AF523, MAB5231, from R&D Systems.

### Reference Level

The methods of the disclosure are based on comparing the measured level of IL-33/sST2 in a sample from the subject with a reference level of IL-33/sST2. Suitably the reference level is a determinant level. Suitably the reference level determines whether or not the subject is likely to respond to, or benefit from, treatment with an IL-33 axis antagonist.

The inventors have realised that a certain level of baseline IL-33/sST2 in a sample from a subject can indicate the likelihood of successful treatment of said subject with an IL-33 axis antagonist such as those described herein. The inventors have found that those subjects having a level of IL-33/sST2 which is greater than or equal to the reference level of IL-33/sST2 in a sample obtained therefrom are more likely to benefit from treatment and respond to treatment with an IL-33 axis antagonist. Suitably therefore the reference level may be regarded as a threshold level. More specifically, the disclosure illustrates that the numerical reduction in death and/or respiratory failure upon treatment with tozorakimab compared to standard of care was greater in hospitalised subjects with respiratory distress diagnosed with COVID-19 that had a higher level of IL-33/sST2 complex.

Suitably therefore the methods of the disclosure may comprise a step of comparing the measured level of IL-33/sST2 to the reference level. Suitably such a step may be automated.

Suitably therefore the methods of the disclosure may comprise a step of determining whether the measured level of IL-33/sST2 is greater than or equal to the reference level. Suitably such a step may be automated.

Suitably, the measured level of IL-33/sST2 is a baseline level of IL-33/sST2. Suitably a baseline serum level of IL-33/sST2.

Suitably the comparing step and/or determining step take place after any measuring step in the methods of the disclosure.

Suitably if the sample contains a level of IL-33/sST2 which is greater than or equal to the reference level then this indicates that the subject is likely to respond to, or is likely to benefit from treatment with an IL-33 axis antagonist. Suitably if the sample contains a level of IL-33/sST2 which is greater than or equal to the reference level then this indicates that the subject should be selected for treatment with an IL-33 axis antagonist. Suitably therefore the methods of the disclosure may further comprise a step of selecting the subject for treatment with an IL-33 axis antagonist, suitably if the sample contains a level of IL-33/sST2 which is greater than or equal to the reference level. Suitably if the sample contains a level of IL-33/sST2 which is greater than or equal to the reference level then this indicates that the subject should be treated with an IL-33 axis antagonist.

Suitably therefore the methods of the invention may further comprise a step of treating the subject with an IL-33 axis antagonist, suitably if the sample contains a level of IL-33/sST2 which is greater than or equal to the reference level. Suitably treating the subject may comprise administering to the subject an effective amount of an IL-33 axis antagonist as is described elsewhere herein.

Suitably if the sample contains a level of IL-33/sST2 which is less than the reference level then this indicates that the subject is not likely to respond to, or is not likely to benefit from, treatment with an IL-33 axis antagonist. Suitably if the sample contains a level of IL-33/sST2 which is less than the reference level then this indicates that the subject should not be selected for treatment with an IL-33 axis antagonist. Suitably if the sample contains a level of IL-33/sST2 which is less than the reference level then this indicates that the subject should not be treated with an IL-33 axis antagonist. Suitably if the sample contains a level of IL-33/sST2 which is less than the reference level then this indicates that the subject has a decreased likelihood of responding to, or benefiting from, said treatment. Suitably a decreased likelihood in comparison to a subject having a level of IL-33/sST2 which is greater than or equal to the reference level.

Suitably a subject having greater than or equal to the reference level of IL-33/sST2 in a sample obtained therefrom may be more likely to respond to, or more likely to benefit from, treatment with an IL-33 axis antagonist than a subject having less than the reference level of IL-33/sST2 in a sample obtained therefrom. Suitably a subject having greater than or equal to the reference level of IL-33/sST2 in a sample obtained therefrom may have an increased likelihood of responding to, or benefiting from, treatment with an IL-33 axis antagonist than a subject having less than the reference level of IL-33/sST2 in a sample obtained therefrom.

Suitably a subject having less than the reference level of IL-33/sST2 in a sample obtained therefrom may be termed a ‘reference subject’. Suitably therefore, a subject having greater than or equal to the reference level of IL-33/sST2 in a sample obtained therefrom may be more likely to respond to, or more likely to benefit from, treatment with an IL-33 axis antagonist than a reference subject.

Suitably the measured level must be greater than or equal to the reference level to indicate treatment, selection of a subject for treatment, or determination of likelihood to respond to, or likelihood of benefiting from, treatment. Suitably the measured level must be greater than the reference level to indicate treatment, selection of a subject for treatment, or determination of likelihood to respond to, or likelihood to benefit from, treatment.

Suitably, ‘likely to respond to’ treatment may mean that the subject will respond to treatment. Suitably, ‘likely to benefit from’ to treatment may mean that the subject will benefit from treatment. Suitably, therefore, if the sample contains a level of IL-33/sST2 which is greater than or equal to the reference level then this indicates that the subject will respond to, or will benefit from, treatment with an IL-33 axis antagonist.

Suitably the reference level is at about 25 picograms per millilitre (pg/ml). Suitably the reference level is about 26 pg/ml, about 27 pg/ml, about 28 pg/ml, about 29 pg/ml, about 30 pg/ml. In one instance, the reference level is about 30 pg/ml. More specifically, in one instance, the reference level is 30.15 pg/ml, and suitably subjects are treated, selected for treatment, or more likely to respond to treatment if the level of IL-33/sST2 in a sample obtained therefrom is greater than or equal to 30.15 pg/ml. In one instance, subjects are treated, selected for treatment, or more likely to respond to treatment if the level of IL-33/sST2 in a sample obtained therefrom is greater than 30.15 pg/ml.

Suitably ‘about’ means that the measured value may vary +/−10% from the stated value. Suitably ‘about’ means that the measured value may vary +/−1%, 2%, 3%, 4%, 5%, 6%, 7%, 8%, 9% or 10% from the stated value.

Suitably treatment or responding to treatment with an IL-33 axis antagonist may comprise any improvement in the subject's physiology. Suitably any improvement in the subject's condition, disease, disorder or infection, suitably any improvement in the subject's respiratory distress. Suitably treatment or responding to treatment with an IL-33 axis antagonist may comprise a reduction in the subject's respiratory distress. Suitably a reduction in respiratory distress may comprise any of the following: a reduction in breathing rate, an increase in blood oxygen concentration, a reduced shortness of breath, an increased blood pressure, a decrease in heart rate, a decrease in chest pain, normal skin colour, a decrease in sweating, a decrease in wheezing, a decrease in confusion, and a decrease in tiredness.

Suitably treatment or responding to treatment with an IL-33 axis antagonist may comprise removal of oxygen or ventilator support from the subject, or a decrease in the number of days the subject requires oxygen or ventilation.

Suitably treatment or responding to treatment with an IL-33 axis antagonist may comprise a clinical improvement of at least 2 points on a 9 point category ordinal scale. Suitably responding to treatment with an IL-33 axis antagonist may comprise reaching a score of 0, 1, or 2 on the ordinal scale.

Suitably treatment or responding to treatment with an IL-33 axis antagonist may comprise reducing the likelihood of death and/or acute respiratory failure, or may comprise preventing respiratory failure and/or death. Suitably reducing the likelihood of death and/or acute respiratory failure may be in comparison to a subject who have not received treatment with an IL-33 axis antagonist. Suitably such that the subject is less likely to experience death and/or acute respiratory failure in comparison to a subject who have not received treatment with an IL-33 axis antagonist. Suitably reducing the likelihood of death and/or acute respiratory failure may be in comparison to subjects who have less than the reference level of IL-33/sST2 in a sample obtained therefrom may be termed a ‘reference subject’. Suitably such that the subject is less likely to experience death and/or acute respiratory failure in comparison to a subject who has less than the reference level of IL-33/sST2 in a sample obtained therefrom.

Suitably treatment or responding to treatment with an IL-33 axis antagonist may comprise reducing the rate of death and/or acute respiratory failure. Suitably reducing the rate of death and/or acute respiratory failure in subjects at risk thereof. Suitably reducing the rate of death and/or acute respiratory failure may be in a population of subjects. Suitably the rate of death and/or acute respiratory failure may be defined as the number of subjects experiencing death and/or acute respiratory failure in a population, suitably a population at risk thereof, suitably a population suffering from respiratory distress. Suitably reducing the rate of death and/or acute respiratory failure may be in comparison to subjects who have not received treatment with an IL-33 axis antagonist. Suitably such that the rate of death and/or acute respiratory failure in subjects receiving the IL-33 axis antagonist treatment in comparison to subjects who have not received the IL-33 axis antagonist treatment is lower. Suitably reducing the rate of death and/or acute respiratory failure may be in comparison to subjects who have less than the reference level of IL-33/sST2 in a sample obtained therefrom may be termed ‘reference subjects’. Suitably such that the rate of death and/or acute respiratory failure in the subjects is lower than subjects having less than the reference level of IL-33/sST2 in a sample obtained therefrom.

Suitably in the context of methods of preventing respiratory distress in a subject, responding to or benefiting from treatment with an IL-33 axis antagonist may be regarded as the prevention of respiratory distress occurring in the subject, suitably such that the subject does not experience respiratory distress. Suitably, in such instances, responding to or benefiting from treatment with an IL-33 axis antagonist may be regarded as the prevention of any one of the symptoms associated with respiratory distress occurring, as defined hereinabove. Suitably, in such instances, responding to or benefiting from treatment with an IL-33 axis antagonist may be regarded as the prevention of all of the symptoms associated with respiratory distress occurring, as defined hereinabove. In some instances of methods of preventing respiratory distress in a subject, responding to or benefiting from treatment with an IL-33 axis antagonist may be regarded as the prevention of acute respiratory failure occurring in the subject, suitably such that the subject does not experience acute respiratory failure. Suitably in the context of methods of preventing acute respiratory failure in a subject, responding to or benefiting from treatment with an IL-33 axis antagonist may be regarded as preventing the need to ventilate or provide oxygen to the subject, suitably preventing the subject requiring ventilation or oxygen. Suitably in such instances, responding to or benefiting from the treatment may be regarded as preventing the need to assist the subject with breathing, suitably preventing the subject requiring any assistance with breathing.

Suitably the response to treatment with an IL-33 axis antagonist occurs within 2 months of first treatment, suitably within about 6 weeks of first treatment, suitably within about 1 month of first treatment, suitably within 29 days of first treatment.

In one instance, responding to treatment with an IL-33 axis antagonist may comprise preventing respiratory failure or death within 29 days of first treatment.

Suitably the phrases ‘respond to’ or ‘benefit from’ may be used interchangeably herein and may indicate the same reactions in a subject as identified above.

### IL-33 Axis Antagonist

The methods of the disclosure comprise treatment of the subject with an IL-33 axis antagonist, or prevention of development of a condition, disease or disorder in the subject with an IL-33 axis antagonist.

‘IL-33 axis antagonist’ as employed herein refers to any agent which attenuates IL-33 activity either directly or indirectly. The antagonist may attenuate the activity of reduced IL-33, oxidised IL-33 or the activity of both. Alternatively, the antagonist may attenuate the activity of other molecules in the IL-33 signalling pathway or ‘axis’, such as ST2, IL1-RaP, MyD88, IRAK1, IRAK4, TRAF6, MAPK, AP-1, NFkB, p38, Foxp3, GATA3, and IRF1. Suitably the IL-33 axis antagonist may attenuate the activity of reduced IL-33, oxidised IL-33, ST2 and/or IL1-RaP.

Suitably the IL-33 axis antagonist is specific to reduced and/or oxidised IL-33, and suitably attenuates the activity of reduced and/or oxidised IL-33. Suitably in such cases, the IL-33 axis antagonist may simply be known as an ‘IL-33 antagonist’.

Suitably the IL-33 axis antagonist is specific to ST2, and suitably attenuates the activity of ST2. Suitably in such cases, the IL-33 axis antagonist may be known as an ‘ST2 antagonist’. An example of a suitable ST2 antagonist is the anti-ST2 antibody astegolimab, also known as MSTT1041A, AMG282 and RG6149.

Suitably the IL-33 axis antagonist is specific to IL1-RaP, and suitably attenuates the activity of IL1-RaP. Suitably in such cases, the IL-33 axis antagonist may be known as an ‘IL1-RaP antagonist’.

Suitably, the attenuation is by binding IL-33 in reduced or oxidised forms, or to ST2, or to IL1-RaP. Suitably, wherein the antagonist attenuates reduced IL-33 activity and oxidised IL-33 activity, the attenuation is by binding to IL-33 in reduced form (i.e. by binding to reduced IL-33).

Suitably, the IL-33 axis antagonist is a binding molecule or fragment thereof. Suitably therefore the IL-33 axis antagonist may be referred to as an IL-33 axis binding antagonist.

A “binding molecule” or “antigen binding molecule” of the present disclosure refers in its broadest sense to a molecule that specifically binds an antigenic determinant. Suitably, the binding molecule specifically binds to IL-33, in particular reduced IL-33 or oxidised IL-33, to ST2 or to IL1 RaP. In one instance, the binding molecule specifically binds reduced IL-33.

Suitably, the binding molecule may be selected from: an antibody, an antigen-binding fragment thereof, an aptamer, at least one heavy or light chain CDR of a reference antibody molecule, and at least six CDRs from one or more reference antibody molecules.

Suitably, the IL-33 axis antagonist is an antibody or binding fragment thereof. Suitably, the IL-33 axis antagonist is an anti-IL-33 antibody or binding fragment thereof, an anti-ST2 antibody or binding fragment thereof, or an anti-IL1 RaP antibody or binding fragment thereof. Suitably, the anti-IL-33 antibody or binding fragment thereof specifically binds to IL-33, in particular reduced IL-33 or oxidised IL-33.

In one instance, the IL-33 axis antagonist is an anti-IL-33 antibody or binding fragment thereof. “Antibody” as employed herein refers to an immunoglobulin molecule as discussed below in more detail, in particular a full-length antibody or a molecule comprising a full-length antibody, for example a DVD-Ig molecule and the like.

A “binding fragment thereof” is interchangeable with “antigen binding fragment thereof” and refers to an epitope/antigen binding fragment of an antibody fragment, for example comprising a binding region, in particular comprising 6 CDRs, such as 3 CDRs in heavy variable region and 3 CDRs in light variable region.

Suitably, the antibody or binding fragment thereof is selected from: naturally-occurring, polyclonal, monoclonal, multispecific, mouse, human, humanized, primatized, or chimeric. Suitably, the antibody or binding fragment thereof may be an epitope-binding fragment, e.g., Fab′ and F(ab′)2, Fd, Fvs, single-chain Fvs (scFv), disulfide-linked Fvs (sdFv), fragments comprising either a VL or VH domain, or fragments produced by a Fab expression library. Suitably, the antibody or binding fragment thereof may be a minibody, a diabody, a triabody, a tetrabody, or a single chain antibody. Suitably, the antibody or binding fragment thereof is a monoclonal antibody. ScFv molecules are known in the art and are described, e.g., in U.S. Pat. No. 5,892,019.

Immunoglobulin or antibody molecules of the disclosure can be of any type (e.g., IgG, IgE, IgM, IgD, IgA, and IgY), class (e.g., IgGI, IgG2, IgG3, IgG4, IgAI, and IgA2, etc.), or subclass of immunoglobulin molecule.

Suitably the IL-33 axis antagonist is a reduced IL-33 antagonist. In other words, the IL-33 axis antagonist attenuates the activity of reduced IL-33. Suitably, the attenuation is by binding to reduced IL-33.

Suitably, the binding molecule or a fragment thereof specifically binds to reduced IL-33 with a binding affinity (Kd) of less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹4 M, 10⁻¹⁴ M, 5×10⁻¹⁵ M, or 10⁻¹⁵ M. Suitably, the binding affinity to reduced IL-33 is less than 5×10⁻¹⁴M (i.e. 0.05 pM). Suitably, the binding affinity is as measured using Kinetic Exclusion Assays (KinExA) or BIACORE™, suitably using KinExA, using protocols such as those described in WO2016/156440 (see e.g., Example 11), which is hereby incorporated by reference in its entirety. Binding molecules that bind to reduced IL-33 with this binding affinity appear to bind tightly enough to reduced IL-33 to prevent dissociation of the binding molecule/reduced IL-33 complex within biologically relevant timescales. Without wishing to be bound by theory, this binding strength is thought to prevent release of the antigen prior to degradation of the antibody/antigen complex in vivo, such that reduced IL-33 is not released

Suitably, the binding molecule or a fragment thereof may specifically bind to reduced IL-33 with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. For example, a binding molecule of the disclosure may bind to reduced IL-33 or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹. Suitably, the k(on) rate is greater than or equal to 10⁷ M⁻¹ sec⁻¹.

Suitably, the binding molecule or a fragment thereof may specifically bind to reduced IL-33 with an off rate (k(off)) of less than or equal to 5×10-1 sec-1, 10-1 sec-1, 5×10-2 sec-1, 10⁻² sec⁻¹, 5×10-3 sec⁻¹ or 10-3 sec⁻¹. For example, a binding molecule of the disclosure may be said to bind to reduced IL-33 or a fragment or variant thereof with an off rate (k(off)) less than or equal to 5×10⁻⁴ sec⁻¹, 10⁻⁴ sec⁻¹, 5×10⁻⁵ sec⁻¹ 10⁻⁵ sec⁻¹, 5×10⁻⁶ sec⁻¹, 10⁻⁶ sec⁻¹, 5×10⁻⁷ sec⁻¹ or 10⁻⁷ sec⁻¹. Suitably, the k(off) rate is less than or equal to 10⁻³ sec⁻¹. IL-33 is an alarmin cytokine released rapidly and in high concentrations in response to inflammatory stimuli.

Suitably, the IL-33 binding molecule may competitively inhibit binding of IL-33 to the binding molecule 33_640087-7B (as described in WO2016/156440), which is also known as tozorakimab and MEDI3506. Suitably, WO2016/156440 describes that 33_640087-7B binds to reduced IL-33 with particularly high affinity. The light chain of tozorakimab has the amino acid sequence set forth in SEQ ID NO:52, and the heavy chain of tozorakimab has the amino acid sequence set forth in SEQ ID NO: 51.

Tozorakimab is a fully human IgG1 monoclonal antibody that is being developed for the treatment of inter alia chronic obstructive pulmonary disease (COPD). Tozorakimab binds to the human reduced form of IL-33 (IL-33red) and prevents binding of IL 33red to its receptor, ST2. Tozorakimab binds human IL-33 with an exceptionally high affinity of approximately 30 fM, and fully neutralises full length and all mature forms of endogenous IL-33red (Scott et al., ERS International Congress 2022, Barcelona (ES), Abstract OA2254). By binding to IL33red, tozorakimab potently inhibits ST2-dependent inflammatory responses in several primary human cells, and in an allergen-driven in vivo model of lung epithelial injury. The oxidised form of IL-33 (IL 33ox) which cannot bind ST2 signals via the RAGE/EGFR pathway. Tozorakimab cannot bind IL-33ox but can prevent the oxidation of IL-33 and IL-33ox-dependent signaling via the RAGE/EGFR complex and mimic the mechanism of action of ST2 (Scott et al., supra). Inhibition of IL-33ox signaling by tozorakimab can improve airway epithelial repair functions and reverse airway epithelial dysfunction in respiratory diseases, including mucus hyper-secretion (Scott et al., supra). Thus, a binding molecule that competitively inhibits binding of IL-33 to the binding molecule 33_640087-7B is highly likely to inhibit both reduced IL-33 and oxidised IL-33 signalling.

A binding molecule or fragment thereof is said to competitively inhibit binding of a reference antibody to a given epitope if it specifically binds to that epitope to the extent that it blocks, to some degree, binding of the reference antibody to the epitope. Competitive inhibition may be determined by any method known in the art, for example, solid phase assays such as competition ELISA assays, Dissociation-Enhanced Lanthanide Fluorescent Immunoassays (DELFIA®, Perkin Elmer), and radioligand binding assays. For example, the skilled person could determine whether a binding molecule or fragment thereof competes for binding to redIL-33 by using an in vitro competitive binding assay, such as a derivation of the HTRF assay described in example 1 of WO2016/156440, which is hereby incorporated by reference. For example, the skilled person could label a recombinant antibody of Table 1 with a donor fluorophore and mix multiple concentrations with fixed concentration samples of acceptor fluorophore labelled-redIL-33. Subsequently, the fluorescence resonance energy transfer between the donor and acceptor fluorophore within each sample can be measured to ascertain binding characteristics. To elucidate competitive binding molecules the skilled person could first mix various concentrations of a test binding molecule with a fixed concentration of the labelled antibody of Table 1. A reduction in the FRET signal when the mixture is incubated with labelled IL-33 in comparison with a labelled antibody-only positive control would indicate competitive binding to IL-33. A binding molecule or fragment thereof may be said to competitively inhibit binding of the reference antibody to a given epitope by at least 90%, at least 80%, at least 70%, at least 60%, or at least 50%.

In some instances, the IL-33 axis binding antagonist is selected from any of the following anti-IL-33 antibodies: 33_640087-7B known as tozorakimab (as described in WO2016/156440), ANB020 known as etokimab (as described in WO2015/106080), 9675P or itepekimab (as described in US2014/0271658), A25-3H04 (as described in US2017/0283494), Ab43 (as described in WO2018/081075), IL33-158 (as described in US2018/0037644), 10C12.38.H6. 87Y.581 IgG4 (as described in WO2016/077381) or binding fragments thereof, each of the documents being incorporated herein by reference. All of these antibodies are referenced in Table 1.

Suitably, the IL-33 axis binding antagonist is an antibody or antigen-binding fragment comprising the complementarity determining regions (CDRs) of a heavy chain variable region (HCVR or VH) and a light chain variable region pair (LCVR or VL) selected from Table 1. Pair 1 corresponds to the HCVR and LCVR sequences of tozorakimab (33_640087-7B) described in WO2016/156440. Pairs 2-7 correspond to HCVR and LCVR sequences of antibodies described in US2014/0271658. Pairs 8-12 correspond to HCVR and LCVR sequences of antibodies described in US2017/0283494. Pair 13 corresponds to the HCVR and LCVR sequences of ANB020, described in WO2015/106080. Pairs 14-16 correspond to HCVR and LCVR sequences of antibodies described in WO2018/081075. Pair 17 corresponds to HCVR and LCVR sequences of IL33-158 described in US2018/0037644. Pair 18 corresponds to HCVR and LCVR sequences of 10C12.38.H6. 87Y.581 IgG4 described in WO2016/077381.

TABLE 1
Exemplary anti-IL-33 antibody HCVR and LCVR pairs

| Pair | SEQ ID NO: | HCVR (VH) sequence | SEQ ID NO: | LCVR (VL) sequence |
|---|---|---|---|---|
| 1 | SEQ ID NO: 1 | EVQLLESGGGLVQPGGSLRL | SEQ ID NO: | SYVLTQPPSVSVSPGQTA |
| | | SCAASGFTFSSYAMSWVRQ | 19 | SITCSGEGMGDKYAAWY |
| | | APGKGLEWVSGISAIDQSTY | | QQKPGQSPVLVIYRDTKR |
| | | YADSVKGRFTISRDNSKNTL | | PSGIPERFSGSNSGNTATL |
| | | YLQMNSLRAEDTAVYYCAR | | TISGTQAMDEADYYCGVI |
| | | QKFMQLWGGGLRYPFGYW | | QDNTGVFGGGTKLTVL |
| | | GQGTMVTVSS | | |
| 2 | SEQ ID NO: 2 | EVQLVESGGGLVQPGGSLR | SEQ ID NO: | DIQMTQSPSSVSASVGDR |
| | | LSCAASGFTFRSFAMSWVR | 20 | VTITCRASQGFSSWLAW |
| | | QAPGKGLELVSDLRTSGGST | | YQQKPGKAPKLLIYAASS |
| | | YYADSVKGRLTISRDNSKNT | | LQSGVPSRFSGSGSGTDF |
| | | LYLQMNSLRAEDTAVYYCA | | TLTITNLQPEDFATYYCQ |
| | | KSHYSTSWFGGFDYWGQGT | | QANSFPLTFGGGTKVEIK |
| | | LVTVSS | | |
| 3 | SEQ ID NO: 3 | QVQLQESGPGLVKPSETLSL | SEQ ID NO: | DIQMTQSPSSVSASVGDR |
| | | TCTVSGGSISSYYWSWIRQP | 21 | VTITCRASQGISTWLAWF |
| | | PGKGLELIGYIYYSGSTNYN | | QQKPGKAPKLLIYAASTL |
| | | PSLKSRVTISVDTSKNHFSLK | | QGGVPSRFSGSGSGPEFT |
| | | LSSVTAADTAVYYCARSQY | | LTISSLQPEDFATYYCQQ |
| | | TSSWYGSFDIWGQGTMVTV | | ANSFPWTFGQGTKVEIK |
| | | SS | | |
| 4 | SEQ ID NO: 4 | QVQLVQSGAEVKKPGASVK | SEQ ID NO: | DIQMTQSPSSVSASVGDR |
| | | VSCKASGYTFNSYGISWVR | 22 | VTITCRASQGFSSWLAW |
| | | QAPGQGLEWMGWISSHNG | | YQQKPGKAPQLLIYAASS |
| | | NSHYVQKFQGRVSMTTDTS | | LQSGVPSRFSGSGSGSDF |
| | | TSTAYMELRSLRSDDTAVY | | TLTISSLQPEDFATYYCQ |
| | | YCARHSYTTSWYGGFDYW | | QANSFPLTFGGGTKVEIK |
| | | GQGTLVTVSS | | |
| 5 | SEQ ID NO: 5 | EVQLVESGGGLVQPGGSLR | SEQ ID NO: | DIQMTQSPSSVSASVGDR |
| | | LSCAASGFTFSSYALTWVRQ | 23 | VTITCRASQGVVSWLAW |
| | | APGKGLEWVSFISGSGGRPF | | YQQKPGKAPKLLIYAASS |
| | | YADSVKGRFTISRDNSKNM | | LQSGVPSRFSGSGSGTDF |
| | | LYLQMNSLRAEDTAIYYCA | | TLTISSLQPEDFATYYCQ |
| | | KSLYTTSWYGGFDSWGQGT | | QSNSFPFTLGPGTKVDIK |
| | | LVTVSS | | |
| 6 | SEQ ID NO: 6 | EVQLVESGGGLVQPGGSLR | SEQ ID NO: | DIQMTQSPSSVSASVGDR |
| | | LSCAASGFTFSNYAMTWVR | 24 | VTITCRASQGISSWLAWY |
| | | QAPGKGLEWVSTISGSGDN | | QQKPGKAPQLLIYAASRL |
| | | TYYADSVQGRFTISRGHSKN | | QSGVPSRFWGSGSGTDFT |
| | | TLYLQMNSLRAEDTAVYYC | | LTISSLQPEDFATYYCQQ |
| | | AKPTYSRSWYGAFDFWGQ | | ANNFPFTFGPGTKVDIK |
| | | GTMVTVSS | | |
| 7 | SEQ ID NO: 7 | EVQLVESGGNLEQPGGSLRL | SEQ ID NO: | DIQMTQSPSSVSASVGDR |
| | | SCTASGFTFSRSAMNWVRR | 25 | VTITCRASQGIFSWLAWY |
| | | APGKGLEWVSGISGSGGRT | | QQKPGKAPKLLIYAASSL |
| | | YYADSVKGRFTISRDNSKNT | | QSGVPSRFSGSGSGTDFT |
| | | LYLQMNSLSAEDTAAYYCA | | LTISSLQPEDFAIYYCQQA |
| | | KDSYTTSWYGGMDVWGHG | | NSVPITFGQGTRLEIK |
| | | TTVTV | | |
| | | SS | | |
| 8 | SEQ ID NO: 8 | EVQLLESGGGLVQPGGSLRL | SEQ ID NO: | QSVLTQPPSASGTPGQRV |
| | | SCAASGFTFSDYYMNWVRQ | 26 | TISCTGSSSNIGAVYDVH |
| | | APGKGLEWVSSISRYSSYIY | | WYQQLPGTAPKLLIYRN |
| | | YADSVKGRFTISRDNSKNTL | | NQRPSGVPDRFSGSKSGT |
| | | YLQMNSLRAEDTAVYYCAR | | SASLAISGLRSEDEADYY |
| | | DIGGMDVWGQGTLVTVSS | | CQTYDSSRWVFGGGTKL |
| | | | | TVL |
| 9 | SEQ ID NO: 9 | EVQLLESGGGLVQPGGSLRL | SEQ ID NO: | QSVLTQPPSASGTPGQRV |
| | | SCAASGFTFSNYYMHWVRQ | 27 | TISCSGSSSNIGNNAVSW |
| | | APGKGLEWVSSISARSRYHY | | YQQLPGTAPKLLIYASNM |
| | | YADSVKGRFTISRDNSKNTL | | RVIGVPDRFSGSKSGTSA |
| | | YLQMNSLRAEDTAVYYCAR | | SLAISGLRSEDEADYYCG |
| | | LATRHNAFDIWGQGTLVTV | | AWDDSQKALVFGGGTKL |
| | | SS | | TVL |
| 10 | SEQ ID NO: 10 | EVQLLESGGGLVQPGGSLRL | SEQ ID NO: | QSVLTQPPSASGTPGQRV |
| | | SCAASGFTFSNYYMHWVRQ | 28 | TISCSGSSSNIGRNAVNW |
| | | APGKGLEWVSSISARSSYIY | | YQQLPGTAPKLLIYASNM |
| | | YADSVKGRFTISRDNSKNTL | | RVSGVPDRFSGSKSGTSA |
| | | YLQMNSLRAEDTAVYYCAR | | SLAISGLRSEDEADYYCW |
| | | LATRNNAFDIWGQGTLVTV | | AWDDSQKVGVFGGGTK |
| | | SS | | LTVL |
| 11 | SEQ ID NO: 11 | EVQLLESGGGLVQPGGSLRL | SEQ ID NO: | QSVLTQPPSASGTPGQRV |
| | | SCAASGFTFSRYYMHWVRQ | 29 | TISCSGSSSNIGRNAVNW |
| | | APGKGLEWVSSISAQSSHIY | | YQQLPGTAPKLLIYASNM |
| | | YADSVEGRFTISRDNSKNTL | | RRSGVPDRFSGSKSGTSA |
| | | YLQMNSLRAEDTAVYYCAR | | SLAISGLRSEDEADYYCS |
| | | LATRQNAFDIWGQGTLVTV | | AWDDSQKVVVFGGGTK |
| | | SS | | LTVL |
| 12 | SEQ ID NO: 12 | EVQLLESGGGLVQPGGSLRL | SEQ ID NO: | QSVLTQPPSASGTPGQRV |
| | | SCAASGFTFSNYYMHWVRQ | 30 | TISCSGSSSNIGNNAVNW |
| | | APGKGLEWVSSISARSSYLY | | YQQLPGTAPKLLIYASNM |
| | | YADSVKGRFTISRDNSKNTL | | RRPGVPDRFSGSKSGTSA |
| | | YLQMNSLRAEDTAVYYCAR | | SLAISGLRSEDEADYYCE |
| | | LATRHVAFDIWGQGTLVTV | | AWDDSQKAVVFGGGTK |
| | | SS | | LTVL |
| 13 | SEQ ID NO: 13 | MRAWIFFLLCLAGRALAQV | SEQ ID NO: | MRA WIFFLLCLAGRALA |
| | | QLMQSGAEVKKPGASVKVS | 31 | DIQLTQSPSFLSASVGDR |
| | | CKASGYTFTSYWMHWVRQ | | VTITCKASQDVGTAVAW |
| | | APGQGLEWMGTIYPRNSNT | | YQQKPGKAPKLLIYWAS |
| | | DYNQKFKARVTMTRDTSTS | | TRHTGVPSRFSGSGSGTE |
| | | TVYMELSSLRSEDTAVYYC | | FTLTISSLQPEDFATYYCQ |
| | | ARPLYYYLTSPPTLFWGQGT | | QAKTYPFTFGSGTKLEIK |
| | | LVTVSS | | R |
| 14 | SEQ ID NO: 14 | EVQLVETGGGLIQPGGSLRL | SEQ ID NO: | EIVLTQSPGTLSLSPGERA |
| | | SCAASGFTFSSYAMSWVRQ | 32 | TLSCRASQSVGINLSWYQ |
| | | APGKGLEWVSAISGSGGSTY | | QKPGQAPRLLIYGASHRA |
| | | YADSVKGRFTISRDNSKNTL | | TGIPDRFSGSGSGTDFTLT |
| | | YLQMNSLRAEDTAVYYCAR | | ISRLEPEDFAVYYCHQYS |
| | | TLHGIRAAYDAFIIWGQGTL | | QSPPFTFGGGTKVEIK |
| | | VTVSS | | |
| 15 | SEQ ID NO: 15 | EVQLVETGGGLIQPGGSLRL | SEQ ID NO: | EIVLTQSPGTLSLSPGERA |
| | | SCAASGFTFSFYAMSWVRQ | 33 | TLSCRASQSVGINLSWYQ |
| | | APGKGLEWVSAISGSGGSTY | | QKPGQAPRLLIYGASHRL |
| | | YADSVKGRFTISRDNSKNTL | | TGIPDRFSGSGSGTDFTLT |
| | | YLQMNSLRAEDTAVYYCAR | | ISRLEPEDFAVYYCHQYS |
| | | TLHGIRAAYDAFIIWGQGTL | | QPPPFTFGGGTKVEIK |
| | | VTVSS | | |
| 16 | SEQ ID NO: 16 | EVQLVETGGGLIQPGGSLRL | SEQ ID NO: | EIVLTQSPGTLSLSPGERA |
| | | SCAASGFTFSFYAMSWVRQ | 34 | TLSCRASQSVGINLSWYQ |
| | | APGKGLEWVSAISGSGGSTY | | QKPGQAPRLLIYGASHRL |
| | | YADSVKGRFTISRDNSKNTL | | TGIPDRFSGSGSGTDFTLT |
| | | YLQMNSLRAEDTAVYYCAR | | ISRLEPEDFAVYYCHQYS |
| | | TIHGIRAAYDAFIIWGQGTL | | QPPPFTFGGGTKVEIK |
| | | VTVSS | | |
| 17 | SEQ ID NO: 17 | EVQLVESGGGLVQPGGSLR | SEQ ID NO: | DIQMTQSPSSLSASVGDR |
| | | LSCAASGFTFSSYWMYWVR | 35 | VTITCKASQNINKHLDW |
| | | QAPGKGLEWVAAITPNAGE | | YQQKPGKAPKLLIYFTNN |
| | | DYYPESVKGRFTISRDNAKN | | LQTGVPSRFSGSGSGTDF |
| | | SLYLQMNSLRAEDTAVYYC | | TLTISSLQPEDFATYYCFQ |
| | | ARGHYYYTSYSLGYWGQG | | YNQGWTFGGGTKVEIK |
| | | TL VTVSS | | |
| 18 | SEQ ID NO: 18 | EVQLVESGGGLVQPGGSLR | SEQ ID NO: | EIVLTQSPATLSLSPGERA |
| | | LSCAASGFTFSSFSMSWVRQ | 36 | TLSCRASESVAKYGLSLL |
| | | APGKGLEWVATISGGKTFT | | NWFQQKPGQPPRLLIFAA |
| | | DYVDSVKGRFTISRDDSKNT | | SNRGSGIPARFSGSGSGT |
| | | LYLQMNSLRAEDTAVYYCT | | DFTLTISSLEPEDFAVYYC |
| | | RANYGNWFFEVWGQGTLV | | QQSKEVPFTFGQGTKVEI |
| | | TVSS | | K |

Suitably the skilled person knows of available methods in the art to identify CDRs within the heavy and light variable regions of an antibody or antigen-binding fragment thereof. Suitably the skilled person may conduct sequence-based annotation, for example. The regions between CDRs are generally highly conserved, and therefore, logic rules can be used to determine CDR location. The skilled person may use a set of sequence-based rules for conventional antibodies (Pantazes and Maranas, Protein Engineering, Design and Selection, 2010), alternatively or additionally he may refine the rules based on a multiple sequence alignment. Alternatively, the skilled person may compare the antibody sequences to a publicly available database operating on Kabat, Chothia or IMGT methods using the BLASTP command of BLAST+ to identify the most similar annotated sequence. Each of these methods has devised a unique residue numbering scheme according to which it numbers the hypervariable region residues and the beginning and ending of each of the six CDRs is then determined according to certain key positions. Upon alignment with the most similar annotated sequence, for example, the CDRs can be extrapolated from the annotated sequence to the non-annotated sequence, thereby identifying the CDRs. Suitable tools/databases are: the Kabat database, Kabatman, Scalinger, IMGT, Abnum for example.

Suitably, the IL-33 axis antagonist is an anti-IL-33 antibody or antigen-binding fragment comprising a heavy chain variable region (HCVR or VH) and light chain variable region (LCVR or VL) pair selected from Table 1.

Suitably therefore in one instance, there is provided a method of treating a subject suffering from respiratory distress, or preventing respiratory distress in a subject at risk thereof, comprising administering to the subject an effective amount of an anti-IL-33 antibody or antigen-binding fragment comprising heavy chain variable region and light chain variable region pair selected from Table 1, preferably 33_640087-7B/tozorakimab, wherein the level of IL-33/sST2 in a sample obtained from the subject is greater than or equal to a reference level of about 25 pg/ml.

Suitably therefore in one instance, there is provided a method of treating a subject suffering from respiratory distress, or preventing respiratory distress in a subject at risk thereof, wherein the method comprises: measuring the level of IL-33/sST2 in a sample obtained from the subject; and administering to the subject an effective amount of an anti-IL-33 antibody or antigen-binding fragment comprising heavy chain variable region and light chain variable region pair selected from Table 1, preferably 33_640087-7B/tozorakimab, if the level of IL-33/sST2 in the sample is greater than or equal to a reference level of about 25 pg/ml.

Suitably therefore in one instance, there is provided a method of selecting a subject suffering from respiratory distress, or at risk of suffering from respiratory distress, for treatment with an anti-IL-33 antibody or antigen-binding fragment comprising a heavy chain variable region and light chain variable region pair selected from Table 1, preferably 33_640087-7B/tozorakimab, comprising: measuring the level of IL-33/sST2 in a sample obtained from the subject; and selecting the subject for said treatment if the level of IL-33/sST2 in the sample is greater than or equal to a reference level of about 25 pg/ml.

Suitably therefore in one instance, there is provided a method of determining whether a subject suffering from respiratory distress, or at risk of suffering from respiratory distress, is likely to respond to treatment with an anti-IL-33 antibody or antigen-binding fragment comprising heavy chain variable region and light chain variable region pair selected from Table 1, preferably 33_640087-7B/tozorakimab, comprising: measuring the level of IL-33/sST2 in a sample obtained from the subject; and determining that the subject is likely to respond to said treatment if the measured level of IL-33/sST2 in the sample is greater than or equal to the reference level.

Suitably, the anti-IL33 antibody or antigen binding fragment thereof comprises a HCVRVH of the sequence of SEQ ID NO:1 and a LCVRVL of the sequence of SEQ ID NO:19.

Suitably, the anti-IL33 antibody or antigen binding fragment thereof comprises a HCVRVH of the sequence of SEQ ID NO:7 and a LCVRVL of the sequence of SEQ ID NO:25.

Suitably, the anti-IL33 antibody or antigen binding fragment thereof comprises a HCVRVH of the sequence of SEQ ID NO:11 and a LCVRVL of the sequence of SEQ ID NO:29.

Suitably, the anti-IL33 antibody or antigen binding fragment thereof comprises a HCVRVH of the sequence of SEQ ID NO:13 and a LCVRVL of the sequence of SEQ ID NO:31.

Suitably, the anti-IL33 antibody or antigen binding fragment thereof comprises a HCVRVH of the sequence of SEQ ID NO:16 and a LCVRVL of the sequence of SEQ ID NO:34.

Suitably, the anti-IL33 antibody or antigen binding fragment thereof comprises a HCVRVH of the sequence of SEQ ID NO:17 and a LCVRVL of the sequence of SEQ ID NO:35.

Suitably, the anti-IL33 antibody or antigen binding fragment thereof comprises a HCVRVH of the sequence of SEQ ID NO:18 and a LCVRVL of the sequence of SEQ ID NO:36.

Suitably, therefore, the IL-33 axis antagonist is a binding molecule which may comprise 3 CDRs, for example in a heavy chain variable region independently selected from SEQ ID NO: 1, 7, 11, 13, 16, 17 and 18.

Suitably the IL-33 axis antagonist is a binding molecule which comprises 3 CDRs in a heavy chain variable region according to SEQ ID NO:1.

Suitably, the IL-33 axis antagonist is a binding molecule which may comprise 3 CDRs in a light chain variable region independently selected from SEQ ID NO: 19, 25, 29, 31, 34, 35 and 36.

Suitably, the IL-33 axis antagonist is a binding molecule which comprises 3 CDRs in a light chain variable region according to SEQ ID NO:19.

Suitably, therefore, the IL-33 axis antagonist is a binding molecule which may comprise 3 CDRs, for example in a heavy chain variable region independently selected from SEQ ID NO: 1, 7, 11, 13, 16, 17 and 18 and 3 CDRs, for example in a light chain variable region independently selected from SEQ ID NO: 19, 25, 29, 31, 34, 35 and 36.

Suitably, therefore the IL-33 axis antagonist is a binding molecule which comprises 3 CDRs in a heavy chain variable region according to SEQ ID NO:1, and 3 CDRs in a light chain variable region according to SEQ ID NO:19.

Suitably, therefore, the IL-33 axis antagonist is a binding molecule which may comprise a variable heavy domain (VH) and a variable light domain (VL) having VH CDRs 1-3 having the sequences SEQ ID NO: 37, 38 and 39, respectively, wherein one or more VHCDRs have 3 or fewer single amino acid substitutions, insertions and/or deletions.

Suitably, therefore, the IL-33 axis antagonist is a binding molecule comprising a VH domain which comprises VHCDRs 1-3 of SEQ ID NO: 37, SEQ ID NO: 38 and SEQ ID NO: 39, respectively.

Suitably, therefore, the IL-33 axis antagonist is a binding molecule comprising a VH domain which comprises VHCDRs 1-3 consisting of SEQ ID NO: 37, SEQ ID NO: 38 and SEQ ID NO: 39, respectively.

Suitably, therefore, the IL-33 antagonist is a binding molecule which may comprise a variable heavy domain (VH) and a variable light domain (VL) having VL CDRs 1-3 having the sequences of SEQ ID NO: 40, 41 and 42, respectively, wherein one or more VLCDRs have 3 or fewer single amino acid substitutions, insertions and/or deletions.

Suitably, therefore, the IL-33 axis antagonist is a binding molecule comprising a VL domain which comprises VLCDRs 1-3 of SEQ ID NO: 40, SEQ ID NO: 41 and SEQ ID NO: 42, respectively.

Suitably, therefore, the IL-33 axis antagonist is a binding molecule comprising a VL domain which comprises VLCDRs 1-3 consisting of SEQ ID NO: 40, SEQ ID NO: 41 and SEQ ID NO: 42, respectively.

Suitably, therefore, the IL-33 axis antagonist is a binding molecule which may comprise a VHCDR1 having the sequence of SEQ ID NO: 37, a VHCDR2 having the sequence of SEQ ID NO: 38, a VHCDR3 having the sequence of SEQ ID NO: 39, a VLCDR1 having the sequence of SEQ ID NO: 40, a VLCDR2 having the sequence of SEQ ID NO: 41, and a VLCDR3 having the sequence of SEQ ID NO: 42.

Suitably, therefore the IL-33 axis antagonist is an antibody or binding fragment thereof comprising a VH and VL, wherein the HCVRVH has an amino acid sequence at least 90%, for example 91, 92, 93, 94, 95, 96, 97, 98, 99 or 100% identical to a HCVRVH according to SEQ ID NO: 1, 7, 11, 13, 16, 17 and 18.

Suitably, therefore the IL-33 axis antagonist is an antibody or binding fragment thereof comprising a VH and VL, wherein the HCVRVH has an amino acid sequence at least 90%, for example 91, 92, 93, 94, 95, 96, 97, 98, 99 or 100% identical to a HCVRVH according to SEQ ID NO: 1.

Suitably, therefore the IL-33 axis antagonist is an antibody or binding fragment thereof comprising a VH and VL, wherein a HCVRVH disclosed above, has a sequence with 1, 2, 3 or 4 amino acids in the framework deleted, inserted and/or independently replaced with a different amino acid.

Suitably, therefore the IL-33 axis antagonist is an antibody or binding fragment thereof comprising a VH and VL, wherein the LCVRVL has an amino acid sequence at least 90%, for example 91, 92, 93, 94, 95, 96, 97, 98, 99 or 100% identical to a LCVRVL according to SEQ ID NO: 19, 25, 29, 31, 34, 35 and 36.

Suitably, therefore the IL-33 axis antagonist is an antibody or binding fragment thereof comprising a VH and VL, wherein the LCVRVL has an amino acid sequence at least 90%, for example 91, 92, 93, 94, 95, 96, 97, 98, 99 or 100% identical to a LCVRVL according to SEQ ID NO: 19.

Suitably, therefore the IL-33 axis antagonist is an antibody or binding fragment thereof comprising a VH and VL, wherein a LCVRVL disclosed above has a sequence with 1, 2, 3 or 4 amino acids in the framework independently deleted, inserted and/or replaced with a different amino acid.

Suitably, therefore the IL-33 axis antagonist is an anti-IL-33 antibody or binding fragment thereof comprising a VH and VL, wherein the VH has an amino acid sequence at least 90%, for example 91, 92, 93, 94, 95, 96, 97, 98, 99 or 100% identical to a HCVRVH according to SEQ ID NO: 1, 7, 11, 13, 16, 17 and 18, and VL has an amino acid sequence at least 90%, for example 91, 92, 93, 94, 95, 96, 97, 98, 99 or 100% identical to a LCVRVL according to SEQ ID NO: 19, 25, 29, 31, 34, 35 and 36.

Suitably, therefore the IL-33 axis antagonist is an anti-IL-33 antibody or binding fragment thereof comprising a VH and VL, wherein the HCVRVH has an amino acid sequence consisting of SEQ ID NO: 1, 7, 11, 13, 16, 17 and 18, and the LCVRVL has an amino acid sequence consisting of SEQ ID NO: 19, 25, 29, 31, 34, 35 and 36.

Suitably, therefore the IL-33 axis antagonist is an anti-IL-33 antibody or binding fragment thereof comprising a VH and VL, wherein the HCVRVH has an amino acid sequence consisting of SEQ ID NO: 1, and the LCVRVL has an amino acid sequence consisting of SEQ ID NO: 19. Suitably in such an instance, the IL-33 axis antagonist is 33_640087-7B, otherwise known as tozorakimab.

### Effective Amount and Administration

The IL-33 axis antagonists in the medical uses and methods described herein may be administered to a subject in the form of a pharmaceutical composition.

Suitably, any references herein to ‘a/the IL-33 axis antagonist’ may also refer to a pharmaceutical composition comprising an/the IL-33 axis antagonist. Suitably the pharmaceutical composition may comprise one or more IL-33 axis antagonists in combination.

Suitably the IL-33 axis antagonist may be administered in a pharmaceutically effective amount for the in vivo treatment of respiratory distress as defined in the medical use and method of treatment aspects herein.

Suitably a ‘pharmaceutically effective amount’ or ‘therapeutically effective amount’ of an IL-33 axis antagonist shall be held to mean an amount sufficient to achieve effective binding to a target molecule within the IL-33 axis and to achieve a benefit, e.g. to ameliorate symptoms of a disease or condition such as respiratory distress as recited in the medical uses/methods herein.

Suitably, the IL-33 axis antagonist or a pharmaceutical composition thereof may be administered to a human or other animal in accordance with the aforementioned methods of treatment/medical uses in an amount sufficient to produce a therapeutic effect.

Suitable doses of the IL-33 axis antagonist or a pharmaceutical composition thereof that may constitute a ‘pharmaceutically effective amount’ or ‘therapeutically effective amount’ may be calculated by the skilled person using known techniques. In one instance, a suitable dose may be about 300 mg.

Suitably, the IL-33 axis antagonist or a pharmaceutical composition thereof can be administered to such human or other animal in a conventional dosage form prepared by combining the IL-33 axis antagonist with one or more conventional pharmaceutically acceptable carriers or diluents according to known techniques.

It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of IL-33 axis antagonists may prove to be particularly effective.

The amount of IL-33 axis antagonist that may be combined with the carrier materials to produce a single dosage form will vary depending upon the subject treated and the particular mode of administration. Suitably, the IL-33 axis antagonist may be administered as a single dose, multiple doses or over an established period of time in an infusion. Suitably, dosage regimens also may be adjusted to provide the optimum desired response (e.g., a therapeutic or prophylactic response).

Suitably, the IL-33 axis antagonist will be formulated so as to facilitate administration and promote stability of the active IL-33 axis antagonist.

Suitably, pharmaceutical compositions are formulated to comprise a pharmaceutically acceptable, non-toxic, sterile carrier such as physiological saline, non-toxic buffers, preservatives and the like.

Suitable formulations for use in the therapeutic methods disclosed herein are described in Remington's Pharmaceutical Sciences (Mack Publishing Co.) 16th ed. (1980).

In some instances, the IL-33 axis antagonist is formulated as a liquid. In some instances the liquid formulation comprises the following: 20 mM L-histidine/L-histidine-hydrochloride, 220 mM L-arginine-hydrochloride, 0.03% (w/v) polysorbate 80, pH 5.5. In some instances, the formulation comprises 150 mg/ml IL-33 axis antagonist.

Methods of administering the IL-33 axis antagonist or a pharmaceutical composition thereof to a subject in need thereof are well known, or are readily determined by those skilled in the art.

Suitably, the route of administration of the IL-33 axis antagonist or pharmaceutical composition thereof may be, for example, oral, parenteral, by inhalation or topical. Suitably, the term parenteral as used herein includes, e.g., intravenous, intraarterial, intraperitoneal, intramuscular, subcutaneous, rectal, or vaginal administration.

Suitably, the IL-33 axis antagonist or pharmaceutical composition thereof may be orally administered in an acceptable dosage form including, e.g., capsules, tablets, aqueous suspensions or solutions.

Suitably, the IL-33 axis antagonist or pharmaceutical composition thereof may be administered by nasal aerosol or inhalation.

In one instance, the IL-33 axis antagonist or pharmaceutical composition thereof is administered intravenously.

In one instance, the IL-33 axis antagonist or pharmaceutical composition thereof is administered at a dose of 300 mg to 600 mg, for example 350 mg, 400 mg, 450 mg, 500 mg, 550 mg, 600 mg. In one instance, the IL-33 axis antagonist or pharmaceutical composition thereof is administered intravenously, suitably at a dose of 300 mg to 600 mg (i.e. a flat dose rather than a body weight-dependent dose is used). In one instance, the IL-33 axis antagonist or pharmaceutical composition thereof is administered intravenously at a dose of 300 mg.

In one instance, the IL-33 axis antagonist or pharmaceutical composition thereof is administered after hospitalization of the subject. In one instance, within 12, 24, 48 or 36 hours of hospitalization of the subject. In one instance, within 36 hours of hospitalization of the subject. Suitably hospitalization of the subject may be regarded as admission to a hospital.

In one instance, the IL-33 axis antagonist or pharmaceutical composition thereof is administered up to about 14 days after the onset of respiratory viral infection symptoms.

The dosage regimen utilised in the present disclosure may comprise administration of only a single dose of the IL-33 axis antagonist or pharmaceutical composition thereof, or may comprise multiple doses (particularly two doses). In a particular instance the therapies of the present disclosure comprise administration of a single dose of the IL-33 axis antagonist or pharmaceutical composition thereof to the subject. That is to say, the methods of treatment disclosed herein comprise administering a single dose of the IL-33 axis antagonist or pharmaceutical composition thereof over a course of therapy.

When multiple doses of the antibody are administered, the doses are suitably spaced, i.e. a gap of an appropriate length is left between doses. In one instance, the IL-33 axis antagonist or pharmaceutical composition thereof is administered about once per week, once every two weeks, once every four weeks, once every 6 weeks, once every 8 weeks, suitably which may be after the first dose. Suitably the first dose may be within 36 hours of hospitalization of the subject. Generally, when multiple doses are administered in the present disclosure, each dose is of the same amount of antibody. In one instance, the IL-33 axis antagonist or pharmaceutical composition thereof is administered in a first dose, and optionally a second dose, wherein the second dose is about two weeks after the first dose. In some instances, the dosage regimen comprises administration of a first dose followed by optional administration of a second dose two weeks later, depending on the clinical condition/progress of the subject. Suitably the first dose may be administrated within 36 hours of hospitalization of the subject. Suitably the first dose may be administered up to about 14 days after the onset of respiratory viral infection symptoms. In one instance, the IL-33 axis antagonist or pharmaceutical composition thereof is administered in a single dose.

In one instance, the IL-33 axis antagonist or pharmaceutical composition thereof is administered intravenously at a dose of 300 mg within 36 hours of hospitalization of the subject. Suitably which may be a single dose.

In one instance, the IL-33 axis antagonist or pharmaceutical composition thereof is administered intravenously at a dose of 300 mg up to about 14 days after the onset of respiratory viral infection symptoms in the subject. Suitably which may be a single dose.

### BRIEF DESCRIPTION OF THE DRAWINGS

Certain elements of the disclosure will now be described, by way of example, with reference to the following drawings, in which:

FIG. 1: shows patient recruitment for the ACCORD phase 2a study during two periods.

FIG. 2: shows the measurement of IL-33/sST2 in patients during the ACCORD Phase 2a study. The numbers of patients treated with SoC versus SoC combined with tozorakimab treatment that died or had respiratory failure at day 29 of the study are shown (a) overall, (b) grouped according to low IL-33/sST2 baseline levels below the reference value or (c) grouped according to high IL-33/sST2 baseline levels equal to or above the reference value.

The disclosure will now be described with reference to the following non-limiting examples, in which:

### EXAMPLES

### Higher Baseline IL-33/sST2 Levels is a Predictive Biomarker to Identify IL-33 Axis Antagonist Responders

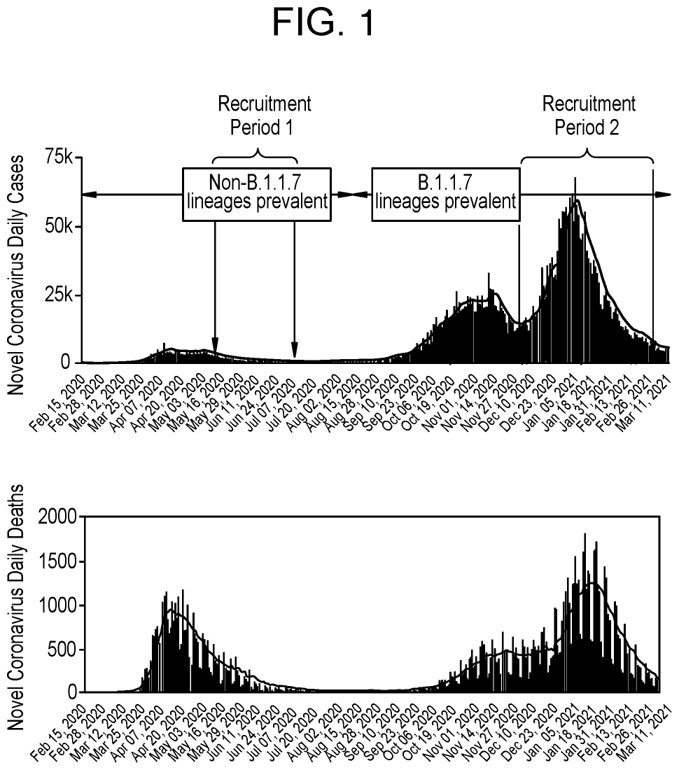

Serum samples from patients with SARS-CoV-2 infection were obtained from patients enrolled in the Phase 2a ACCORD study (EudraCT Number: 2020-001736-95, Wilkinson et al., 2020). Patients were recruited during two time periods across the pandemic as shown in FIG. 1.

Patients were randomized and either received the standard of care (SoC) alone or in combination with an anti-IL-33 antibody (tozorakimab) treatment (300 mg IV and optional second dose if invasively ventilated at day 15) across a 29 day period. The 300 mg IV dose was administered by the following procedure: 2 ml of tozorakimab was diluted with 8 ml of saline to a total volume of 10 ml and administered to the patient via IV push over 1-2 minutes using IV-line filter and followed by IV flush with 5 ml of normal saline.

The SoC varied across the two periods of the ACCORD study as understanding of the SARS-CoV-2 virus developed. The SoC used during each period is shown below in Table 2. The SoC therapy in the second period of the ACCORD study more accurately reflects current best practice.

TABLE 2
SoC
ACCORD: Key SoC Therapies by Period

| | Period 1 | | Period 2 | |
|---|---|---|---|---|
| | (20 May 2020 | | (8 Dec. 2020 | |
| | to 24 Jul. 2020) | | to 2 Mar. 2021) | |
| | SoC | Tozorakimab | SoC | Tozorakimab |
| | (N = 12) | (N = 4) | (N = 32) | (N = 50) |
| Remdesivir | 17% | 25% | 56% | 36% |
| Dexamethasone | 58% | 75% | 100% | 94% |
| Tocilizumab | 0% | 0% | 16% | 12% |

Serum from patients dosed with tozorakimab+standard of care (n=46 as baseline, n=37 at day5, n=10 at day10) and SoC alone (n=37 baseline, n=23 at day 5, n=10 at day 10) were measured for IL-33/sST2 at 1:4 dilution.

All serum samples were stored aliquoted at −80 C after collection.

MSD S-PLEX Assays® were performed on the serum samples obtained from the patients at MSD (Gaithersburg, USA) using S-PLEX® technology using the antibody pair described in Table 3 to measure the levels of IL-33/sST2 in the serum.

Note that any other assay method available in the art, as explained hereinabove, could have been used to measure the levels of IL-33/sST2 in the serum samples.

TABLE 3
Antibody pair for IL-33/sST2 assays

| | Binding Molecule | Reporter Molecule |
|---|---|---|
| Assay | (Capture Ab) | (Detection Ab) |
| IL-33/ | AB1070008 (see sequences | R&D systems |
| sST2 | described elsewhere herein) | MAB5232 |

TABLE 4
Estimated lower limit of detection (LLOD)
of MSD S-PLEX assays ®

| IL-33 assay format | MSD S-PLEX assay ® |
|---|---|
| | Estimated LLOD (pg/ml) |
| IL-33/sST2 complex | 0.370 |

Estimated LLOD is calculated as concentration off the standard curve which produced signals which are 2.5 standard deviations above the diluent only (buffer only).

The custom MSD S-PLEX IL-33/sST2 complex assay was prepared and validated by Mesoscale Discovery (MSD) and performed according to manufacturer's instructions. All incubations required plate shaking at room temperature (RT) unless otherwise stated. Plates were washed where stated with 3× with wash buffer (phosphate buffered saline (PBS)/0.05% Tween-20). Biotin coating capture mAb (Table 5) was diluted in Diluent 100 (MSD) with S-Plex coating reagent (Table 5). Assay plates (MSD) were coated with 50 μl/well of coating solution and incubated for 1 h. Blocking reagent (1×final concentration) was prepared by diluting S-Plex Blocker reagent (MSD) in Diluent 101 (MSD). Recombinant IL-33/sST2 complex standard curve was generated by diluting stock to top standard concentration in Diluent 100 (Table 5) and performing 4-fold serial dilutions. 25 μl of blocking reagent was added to all wells and 25 μl of standards and samples were added to wells and incubated for 1.5 h. TURBO-boost solution was prepared by diluting TURBO-boost labelled detection mAb stock (Table 5) to working concentration in Diluent 3 (MSD). Plates were washed and 50 μl of TURBO-boost detection mAb solution added per well and incubated for 1 h. Plates were washed and 50 μl of enhancement solution (MSD) added/well and incubated for 30 min. Plates were washed and 50 μl of detection solution (MSD) added/well and incubated at 26° C. for 1 h. Plates washed and 150 μl of 1×Read BufferA (MSD) added/well. Plates were read on an MSD MESO SECTOR S600 instrument.

TABLE 5
Concentrations of antibodies and IL-33/sST2 standard

| Reagent | IL-33/ST2 complex assay |
|---|---|
| Biotin coating capture Ab (μg/ml) | 0.25 |
| TURBO-boost detection Ab (μg/ml) | 0.1 |
| Top standard (Units/ml)* | 1000 |

*Data is expressed in Units/ml for IL-33/sST2 complex; this can be approximated to pg/ml if you assume 100% complex formation for IL-33/sST2 standard.

The MSD S-PLEX assay was sensitive enough to determine pg/ml levels of IL-33/sST2 in the serum of patients.

A subgroup analysis of the endpoint Death or Respiratory Failure at Day 29′ was performed, based on median value for baseline IL-33/sST2. The median value at baseline was 30.15 U/ml and the subgroups were defined as IL-33 low: Baseline IL-33/sST2<30.15 U/ml; IL-33 high: Baseline IL-33/sST2>=30.15 U/m.

The proportion of subjects who died by, or who were in respiratory failure at Day 29 was calculated for each treatment in each subgroup, and the relative risk (tozorakimab:placebo) was calculated for each subgroup together with 80% confidence interval. Additionally, a logistic regression model was fitted for each subgroup, adjusting for age (continuous) and baseline severity (WHO score of 3 or 4 versus 5). From the logistic regression model, the odds ratio (tozorakimab:placebo) of being dead or in respiratory failure at Day 29 was calculated for each subgroup, with 80% confidence interval.

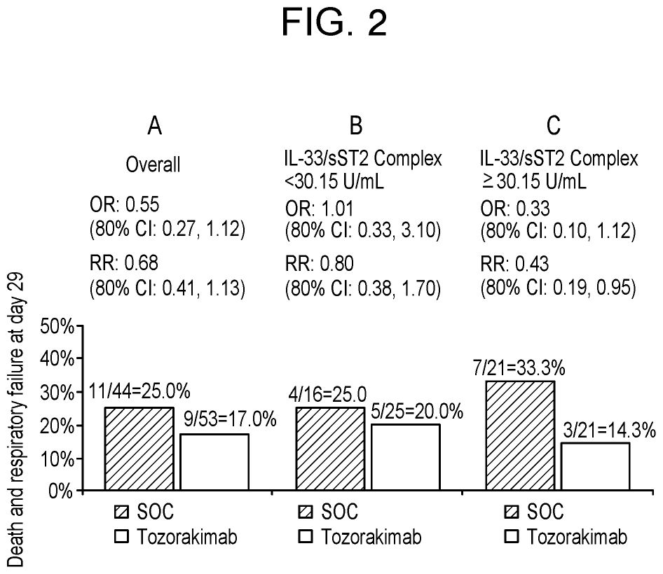

Using the assay, it was determined that those patients having a high baseline serum level of IL-33/sST2, of equal to or above 30.15 pg/ml, had a much reduced likelihood of death or respiratory failure after 29 days when treated with tozorakimab as shown in Table 6 and FIG. 2.

Such patients form a sub-group which are likely to respond well to IL-33 axis antagonist treatments, thereby providing a way of stratifying and selecting patients in respiratory distress for treatment with IL-33 axis antagonists. The treatments may be well applicable to subjects in respiratory distress hospitalized with viral lower respiratory tract infection requiring supplemental oxygen. These subjects are particularly at risk of dying and.or progressing to invasive mechanical ventilation (IMV) or ECMO.

TABLE 6

| | | | | | tozorakimab + | |
|---|---|---|---|---|---|---|
| | | | SoC | | SoC | |
| Sub-group | | | (n = 16) | | (n = 25) | |
| IL-33/ | Death or | Yes: n(%) | 4 | (25%) | 5 | (20%) |
| sST2 <30.15 | respiratory | No: n(%) | 12 | (75%) | 20 | (80%) |
| pg/ml | failure at | | | | | |
| | day 29 | | | | | |
| | Risk ratio | | N/A | | 0.8 | |
| | (80% CI)* | | | | (0.38, 1.70) | |
| | Odds ratio | | N/A | | 1.01 | |
| | (80% CI)* | | | | (0.33, 3.10) | |
| | | | | | tozorakimab + | |
| | | | SoC | | SoC | |
| | | | (n = 21) | | (n = 21) | |
| IL-33/ | Death or | Yes: n(%) | 7 | (33%) | 3 | (14%) |
| sST2 ≥30.15 | respiratory | No: n(%) | 14 | (67%) | 18 | (86%) |

| | | | |
|---|---|---|---|
| pg/ml | failure at | | |
| | day 29 | | |
| | Risk ratio | N/A | 0.43 |
| | (80% CI)* | | (0.19, 0.95) |
| | Odds ratio | N/A | 0.33 |
| | (80% CI)* | | (0.10, 1.12) |

Sequences Additional to those in Table 1

IL-33 axis antagonist binding molecule (tozorakimab):
VH CDRs 1-3:
VH CDR1 SEQ ID NO 37:
SYAMS
VH CDR2 SEQ ID NO 38:
GISAIDQSTYYADSVKG
VH CDR3 SEQ ID NO 39:
QKFMQLWGGGLRYPFGY
VL CDRs 1-3:
VL CDR1 SEQ ID NO 40:
SGEGMGDKYAA
VL CDR2 SEQ ID NO 41:
RDTKRPS
VL CDR3 SEQ ID NO 42:
GVIQDNTGV
Anti-IL-33/sST2 antibody (AB1070008):
AB1070008 VH according to
SEQ ID NO: 43
QVQLQQSGPELVKPGASVKTSCKASGYSFTSYYIHWVKQRPGQGLEWIGWIYPGSGNTKY
NEKFKGKATLTADTSSSTAFMQLSSLTSEDSAVYYCASGFHYYGRMDYWGQGTTLTVSS
AB1070008 VL according to
SEQ ID NO: 44
DIQMTQSPSSLSASLGERVSLTCRASQEISGYLSWLQQKPDGTIKRLIYSTSTLDSGVPKSF
SGSRSGSDYSLTISSLESEDFADYYCLQYASSPWTFGGGTKLEIK
AB1070008 VHCDRs 1-3:
VH CDR1 SEQ ID NO: 45:
SYYIH
VH CDR2 SEQ ID NO: 46
WIYPGSGNTKYNEKFK
VH CDR3 SEQ ID NO: 47
GFHYYGRMDY
AB1070008 VLCDRs 1-3:
VL CDR1 SEQ ID NO: 48:
RASQEISGYLS
VL CDR2 SEQ ID NO: 49:
STSTLDS
VL CDR3 SEQ ID NO: 50:
LQYASSPWT
Tozorakimab Heavy Chain sequence (SEQ ID NO: 51):
EVQLLESGGGLVQPGGSLRLSCAASGFTFSSYAMSWVRQAPGKGLEWVSGISAIDQSTYY
ADSVKGRFTISRDNSKNTLYLQMNSLRAEDTAVYYCARQKFMQLWGGGLRYPFGYWGQG
TMVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCPAP
ELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
Tozorakimab Light Chain sequence (SEQ ID NO: 52):
SYVLTQPPSVSVSPGQTASITCSGEGMGDKYAAWYQQKPGQSPVLVIYRDTKRPSGIPERF
SGSNSGNTATLTISGTQAMDEADYYCGVIQDNTGVFGGGTKLTVLGQPKAAPSVTLFPPSS
EELQANKATLVCLISDFYPGAVTVAWKADSSPVKAGVETTTPSKQSNNKYAASSYLSLTPE
QWKSHRSYSCQVTHEGSTVEKTVAPTECS

1. A method of treating a subject suffering from respiratory distress, or preventing respiratory distress in a subject at risk thereof, comprising administering to the subject an effective amount of an anti-IL-33 antibody or antigen binding fragment thereof, wherein the level of IL-33/sST2 in a sample obtained from the subject has been determined to be greater than or equal to a reference level of 25 μg/ml, wherein the anti-IL-33 antibody or antigen binding fragment thereof comprises a heavy chain variable region (VH) having VHCDRs 1-3 of SEQ ID NO: 37, SEQ ID NO: 38 and SEQ ID NO: 39, respectively, and a light chain variable region (VL) having VLCDRs 1-3 of SEQ ID NO: 40, SEQ ID NO: 41 and SEQ ID NO: 42, respectively.

2. A method of preventing acute respiratory failure in a subject at risk thereof, comprising administering to the subject an effective amount of an anti-IL-33 antibody or antigen binding fragment thereof, wherein the level of IL-33/sST2 in a sample obtained from the subject has been determined to be greater than or equal to a reference level of 25 μg/ml, wherein the anti-IL-33 antibody or antigen binding fragment thereof comprises a heavy chain variable region having VHCDRs 1-3 of SEQ ID NO: 37, SEQ ID NO: 38 and SEQ ID NO: 39, respectively, and a light chain variable region having VLCDRs 1-3 of SEQ ID NO: 40, SEQ ID NO: 41 and SEQ ID NO: 42, respectively.

3. A method according to claims 1 or 2, further comprising the steps of measuring the level of IL-33/sST2 in a sample obtained from the subject, and administering to the subject an effective amount of the anti-IL-33 antibody or antigen binding fragment thereof if the level of IL-33/sST2 in the sample obtained from the subject is greater than or equal to a reference level of 25 pg/ml.

4. A method of selecting a subject suffering from respiratory distress, or at risk of suffering from respiratory distress, for treatment with an anti-IL-33 antibody or antigen binding fragment thereof comprising: measuring the level of IL-33/sST2 in a sample obtained from the subject; and selecting the subject for said treatment if the level of IL-33/sST2 in the sample is greater than or equal to a reference level of 25 μg/ml, wherein the anti-IL-33 antibody or fragment thereof comprises a heavy chain variable region having VHCDRs 1-3 of SEQ ID NO: 37, SEQ ID NO: 38 and SEQ ID NO: 39, respectively, and a light chain variable region having VLCDRs 1-3 of SEQ ID NO: 40, SEQ ID NO: 41 and SEQ ID NO: 42, respectively.

5. A method of determining whether a subject suffering from respiratory distress, or at risk of suffering from respiratory distress, is likely to respond to treatment with an anti-IL-33 antibody or antigen binding fragment thereof comprising: measuring the level of IL-33/sST2 in a sample obtained from the subject; and determining that the subject is likely to respond to said treatment if the measured level of IL-33/sST2 in the sample is greater than or equal to a reference level of 25 pg/ml, wherein the anti-IL-33 antibody or fragment thereof comprises a heavy chain variable region having VHCDRs 1-3 of SEQ ID NO: 37, SEQ ID NO: 38 and SEQ ID NO: 39, respectively, and a light chain variable region having VLCDRs 1-3 of SEQ ID NO: 40, SEQ ID NO: 41 and SEQ ID NO: 42, respectively.

6. The method of any preceding claim wherein the reference level is 26 pg/ml, 27 pg/ml, 28 pg/ml, 29 pg/ml or 30 pg/ml, preferably wherein the reference level is 30.15 pg/ml.

7. The method of any preceding claim wherein the sample is a whole blood sample, a serum sample, a plasma sample or a combination thereof.

8. The method of claim 7, wherein the sample is a serum sample.

9. The method of any preceding claim wherein the respiratory distress is acute respiratory failure.

10. The method of claim 9, wherein the acute respiratory failure is Type 1 or Type 2 acute respiratory failure.

11. The method of claim 10, wherein the acute respiratory failure is Type 1 acute respiratory failure.

12. The method of any of claims 9 to 11, wherein the acute respiratory failure is caused by a disease, disorder, condition or infection, selected from pneumonia, acute respiratory distress syndrome (ARDS), chronic obstructive pulmonary disease (COPD), asthma, bronchitis, bronchiectasis, emphysema, heart failure, myocardial ischemia, mitral stenosis, pulmonary oedema, pulmonary embolism, thromboembolism, cystic fibrosis, amylotophic lateral sclerosis, muscular dystrophy, Guillain-Barre syndrome, myasthenia gravis, poliomyelitis, polymyositis, botulism, hypokalemia, hypophosphatemia, myxedema, hypothyroidism, sepsis, stroke, acute pancreatitis, transfusion, reperfusion, drug or alcohol overdose, acute lung injury, trauma to the chest, viral or bacterial infection, inhalation injury (from inhaling smoke, fumes, or chemicals), aspiration, and near drowning.

13. The method of any of claims 9 to 11 wherein the acute respiratory failure is caused by a bacterial, fungal, or viral infection, preferably a viral respiratory infection, more preferably a SARS-Cov-2 infection.

14. The method of any preceding claim wherein the subject is suffering from COVID-19.

15. The method of any preceding claim, wherein the subject is suffering from pneumonia, or is suspected from suffering from pneumonia.

16. The method of any preceding claim, wherein the subject is suffering from viral pneumonia, or is suspected from suffering from viral pneumonia.

17. The method of claim 15 or 16, wherein the viral pneumonia is caused by influenza virus A, influenza virus B, respiratory syncytial virus, human parainfluenza virus, adenovirus, metapneumovirus, SARS-COV, Middle East respiratory syndrome virus (MERS-CoV), hantavirus, herpes simplex virus, varicella-zoster virus, measles virus, rubella virus, cytomegalovirus or smallpox virus or dengue virus.

18. The method of claim 17, wherein the viral pneumonia is caused by influenza virus A, influenza virus B, respiratory syncytial virus or human parainfluenza virus.

19. The method of any preceding claim, wherein the subject is suffering from, or is at risk of, acute respiratory failure caused by COVID-19 and/or viral pneumonia.

20. The method of any preceding claim, wherein the subject is suffering from, or is at risk of, type 1 or type 2 acute respiratory failure caused by COVID-19 and/or viral pneumonia.

21. The method of any preceding claim, wherein the subject has tested positive for SARS-Cov-2 infection.

22. The method of any preceding claim, wherein the subject has a viral lower respiratory tract infection or disease.

23. The method according to any preceding claim, wherein the anti-IL-33 antibody or antigen binding fragment thereof comprises a heavy chain variable region (VH) according to SEQ ID NO:1 and a light chain variable region (VL) according to SEQ ID NO.19.

24. The method according to any preceding claim, wherein the IL-33 axis antagonist is tozorakimab.

25. The method of any preceding claim wherein the subject requires supplemental oxygen or ventilation.

26. The method of any preceding claim, wherein the subject is hospitalized.

27. The method of any preceding claim, wherein the subject has a viral lower respiratory tract infection or disease, is hospitalized and requires supplemental oxygen or ventilation.

28. The method of any preceding claim, wherein the subject is hospitalized prior to the method of any of claims 1 to 27.

29. The method according to any of claims 3-28 wherein measuring the level of IL-33/sST2 in a sample obtained from the subject comprises performing an assay on the sample obtained from the subject to determine the level of IL-33/sST2.

30. The method according to claim 29, wherein the assay is an immunoassay.

31. The method according to any of claims 3-30 wherein measuring the level of IL-33/sST2 in a sample obtained from the subject comprises the steps of (i) contacting the sample with one or more binding molecules capable of binding to IL-33/sST2 under conditions sufficient to form complexes and (ii) detecting the level of IL-33/sST2 complexes in the sample.

32. The method according to claim 31, further comprising step (iii) contacting the complexes with one or more reporter molecules capable of binding to the one or more complexes.

33. The method according to claim 31 or 32, wherein the one or more binding molecules, and the one or more reporter molecules are antibodies or antigen binding fragments thereof.

34. The method according to claim 33 wherein the one or more binding molecules are anti-IL-33/sST2 antibodies or antigen binding fragments thereof, and wherein the one or more reporter molecules are anti-IL-33/sST2-binding molecule complex antibodies or antigen binding fragments thereof.

35. The method according to claim 34 wherein the anti-IL-33/sST2 antibodies or antigen binding fragments thereof comprise a heavy chain variable region having VHCDRs 1-3 of SEQ ID NO: 45, SEQ ID NO: 46 and SEQ ID NO: 47, respectively, and a light chain variable region having VLCDRs 1-3 of SEQ ID NO: 48, SEQ ID NO: 49 and SEQ ID NO: 50, respectively.

36. The method according to claim 34 or 35, wherein the anti-IL-33/sST2 antibodies or antigen binding fragments thereof comprise a heavy chain variable region (VH) according to SEQ ID NO: 43 and a light chain variable region (VL) according to SEQ ID NO.44.

37. A method of reducing the likelihood of death and/or acute respiratory failure in a subject suffering from respiratory distress or at risk of suffering from respiratory distress, comprising administering to the subject an effective amount of an anti-IL-33 antibody or antigen binding fragment thereof, wherein the level of IL-33/sST2 in a sample obtained from the subject has been determined to be greater than or equal to a reference level of 25 μg/ml, wherein the anti-IL-33 antibody or antigen binding fragment thereof comprises a heavy chain variable region having VHCDRs 1-3 of SEQ ID NO: 37, SEQ ID NO: 38 and SEQ ID NO: 39, respectively, and a light chain variable region having VLCDRs 1-3 of SEQ ID NO: 40, SEQ ID NO: 41 and SEQ ID NO: 42, respectively.

38. A method according to claim 37 wherein the subject has a viral lower respiratory tract infection or disease, is hospitalized and requires supplemental oxygen or ventilation.
